(19) United States
(12) Patent Application Publication
(10) Pub. No.: US 2015/0025384 A1
(43) Pub. Date: Jan. 22, 2015

(54) ULTRASOUND MEDICAL APPARATUS AND ULTRASOUND DIAGNOSIS APPARATUS

(71) Applicants: Kabushiki Kaisha Toshiba, Minato-ku (JP); Toshiba Medical Systems Corporation, Otawara-shi (JP)

(72) Inventor: Hiroshi ASAHINA, Nasushiobara-shi (JP)

(73) Assignees: Kabushiki Kaisha Toshiba, Minato-ku (JP); Toshiba Medical Systems Corporation, Otawara-shi (JP)

(21) Appl. No.: 14/335,321

(22) Filed: Jul. 18, 2014

(30) Foreign Application Priority Data
Jul. 19, 2013 (JP) 2013-150147

(51) Int. Cl. A61B 8/08; A61B 8/14; A61B 8/12

(52) U.S. Cl. CPC A61B 8/5207; A61B 8/12; A61B 8/14
USPC 600/443

(57) ABSTRACT

In embodiments of an ultrasound medical apparatus, main body part includes ultrasound transducer and is inserted into lumen of subject. First and second blocking parts are inserted into lumen and capable of substantially occluding lumen by changing dimensions. First blocking part is arranged at the opposite side to insertion opening across main body part, and is provided, on surface of insertion opening side, with shieldable opening. Second blocking part is arranged at insertion opening side across main body part, and is provided with path penetrating itself. Fluid supplying part supplies fluid in a state in which its tip part is arranged inside first blocking part through opening to enlarge dimension, and supplies fluid in a state in which tip part is arranged in path to enlarge dimension of second blocking part. Liquid supplying part supplies liquid into space between first and second blocking parts.

### CROSS-REFERENCE TO RELATED APPLICATION

This application is based upon and claims the benefit of priority from Japanese Patent Application No. 2013-150147, filed Jul. 19, 2013; the entire contents of which are incorporated herein by reference.

### FIELD

Embodiments described herein relate to an ultrasound medical apparatus and an ultrasound diagnosis apparatus.

### BACKGROUND

An ultrasound diagnosis apparatus scans the inside of a subject using an ultrasound probe and creates images of the inside of the subject based on echo signals generated from reflected waves.

Transesophageal Echocardiography (TEE) is one example of an ultrasound probe used in an ultrasound diagnosis apparatus. The TEE probe is, for example, orally inserted into an upper gastro tube such as the esophagus, stomach, etc. in order to acquire images of the heart, etc. via the esophagus wall or stomach wall. The TEE probe comprises the following elements: a main body part that is inserted into the upper gastro tube and transmits and receives ultrasound waves; a guiding hollow part that holds the main body part at the tip thereof, is inserted into the esophagus and is capable of manipulating the bending angle; an operation part for manipulating the bending angle of the guiding hollow part; and a connector part for connecting the TEE probe to the main body of the ultrasound diagnosis apparatus. An ultrasound transducer is provided at the tip of the main body part of the TEE probe. By acquiring images of the heart etc. from the lumen of a subject using the TEE probe, high quality images of the heart etc. may be acquired without being affected by bones or subcutaneous fat.

Furthermore, an ultrasound medical apparatus (ultrasound probe) of so-called capsule type has also been proposed. This ultrasound medical apparatus (capsule ultrasound endoscope) comprises an ultrasound transducer and is orally introduced into the esophagus for positioning. The capsule ultrasound endoscope is secured in the esophagus by inflating, with liquid, a balloon provided in the surrounding area thereof. Furthermore, the position of the ultrasound transducer may be adjusted in accordance with inflation of the balloon.

### BRIEF DESCRIPTION OF THE DRAWINGS

FIG. 1 is a schematic diagram illustrating a configuration example of an ultrasound diagnosis apparatus according to an embodiment.

FIG. 2 is a schematic diagram illustrating a configuration example of an ultrasound diagnosis apparatus according to an embodiment.

FIG. 3A is a schematic diagram illustrating a configuration example of an ultrasound diagnosis apparatus according to an embodiment.

FIG. 3B is a schematic diagram illustrating a configuration example of an ultrasound diagnosis apparatus according to an embodiment.

FIG. 4 is a schematic diagram illustrating a configuration example of an ultrasound diagnosis apparatus according to an embodiment.

FIG. 5A is a schematic diagram illustrating a configuration example of an ultrasound diagnosis apparatus according to an embodiment.

FIG. 5B is a schematic diagram illustrating a configuration example of an ultrasound diagnosis apparatus according to an embodiment.

FIG. 6 is a schematic diagram illustrating a configuration example of an ultrasound diagnosis apparatus according to an embodiment.

FIG. 7 is a schematic diagram illustrating a configuration example of an ultrasound diagnosis apparatus according to an embodiment.

FIG. 8 is a flowchart showing an example of an application mode of an ultrasound diagnosis apparatus according to an embodiment.

FIG. 9A is a schematic diagram for explaining an application mode of an ultrasound diagnosis apparatus according to an embodiment.

FIG. 9B is a schematic diagram for explaining an application mode of an ultrasound diagnosis apparatus according to an embodiment.

FIG. 9C is a schematic diagram for explaining an application mode of an ultrasound diagnosis apparatus according to an embodiment.

FIG. 9D is a schematic diagram for explaining an application mode of an ultrasound diagnosis apparatus according to an embodiment.

FIG. 9E is a schematic diagram for explaining an application mode of an ultrasound diagnosis apparatus according to an embodiment.

FIG. 9F is a schematic diagram for explaining an application mode of an ultrasound diagnosis apparatus according to an embodiment.

FIG. 10 is a schematic diagram illustrating a configuration example of an ultrasound diagnosis apparatus according to a modified example.

FIG. 11 is a schematic diagram illustrating a configuration example of an ultrasound diagnosis apparatus according to a modified example.

FIG. 12A is a schematic diagram illustrating a configuration example of an ultrasound diagnosis apparatus according to a modified example.

FIG. 12B is a schematic diagram illustrating a configuration example of an ultrasound diagnosis apparatus according to a modified example.

FIG. 12C is a schematic diagram for explaining an application mode of an ultrasound diagnosis apparatus according to a modified example.

FIG. 12D is a schematic diagram for explaining an application mode of an ultrasound diagnosis apparatus according to a modified example.

FIG. 12E is a schematic diagram for explaining an application mode of an ultrasound diagnosis apparatus according to a modified example.

FIG. 12F is a schematic diagram for explaining an application mode of an ultrasound diagnosis apparatus according to a modified example.

FIG. 12G is a schematic diagram for explaining an application mode of an ultrasound diagnosis apparatus according to a modified example.

FIG. 12H is a schematic diagram for explaining an application mode of an ultrasound diagnosis apparatus according to a modified example.

FIG. 13 is a schematic diagram illustrating a configuration example of an ultrasound diagnosis apparatus according to a modified example.

FIG. 14 is a schematic diagram illustrating a configuration example of an ultrasound diagnosis apparatus according to an embodiment.

FIG. 15 is a schematic diagram illustrating a configuration example of an ultrasound diagnosis apparatus according to an embodiment.

FIG. 16 is a schematic diagram illustrating a configuration example of an ultrasound diagnosis apparatus according to an embodiment.

FIG. 17 is a flowchart showing an example of an application mode of an ultrasound diagnosis apparatus according to an embodiment.

FIG. 18A is a schematic diagram for explaining an application mode of an ultrasound diagnosis apparatus according to an embodiment.

FIG. 18B is a schematic diagram for explaining an application mode of an ultrasound diagnosis apparatus according to an embodiment.

FIG. 18C is a schematic diagram for explaining an application mode of an ultrasound diagnosis apparatus according to an embodiment.

FIG. 18D is a schematic diagram for explaining an application mode of an ultrasound diagnosis apparatus according to an embodiment.

FIG. 18E is a schematic diagram for explaining an application mode of an ultrasound diagnosis apparatus according to an embodiment.

FIG. 18F is a schematic diagram for explaining an application mode of an ultrasound diagnosis apparatus according to an embodiment.

FIG. 18G is a schematic diagram for explaining an application mode of an ultrasound diagnosis apparatus according to an embodiment.

FIG. 18H is a schematic diagram for explaining an application mode of an ultrasound diagnosis apparatus according to an embodiment.

FIG. 18I is a schematic diagram for explaining an application mode of an ultrasound diagnosis apparatus according to an embodiment.

FIG. 18J is a schematic diagram for explaining an application mode of an ultrasound diagnosis apparatus according to an embodiment.

### DETAILED DESCRIPTION

An ultrasound medical apparatus according to one example of embodiments includes a main body part, a first blocking part, a second blocking part, a fluid supplying part and a liquid supplying part. The main body part includes an ultrasound transducer configured to transmit and receive ultrasonic waves, and is inserted into a lumen of a subject. The first blocking part is inserted into the lumen, is arranged at the opposite side to an insertion opening of the subject across the main body part, and is capable of substantially occluding the lumen by changing the dimension thereof. Further, a first opening that can be shielded is provided on a surface of the insertion opening side of the first blocking part. The second blocking part is inserted into the lumen, is arranged at the insertion opening side across the main body part, and is capable of substantially occluding the lumen by changing the dimension thereof. Further, the second blocking part is provided with a first path connecting a second opening that can be shielded and is provided on a surface of the insertion opening side and a third opening that can be shielded and is provided on the opposite surface thereto. The fluid supplying part is configured such that its tip part is removably inserted into the first opening, and supplies fluid to enlarge the dimension of the first blocking part in a state in which the tip part is arranged inside the first blocking part through the first opening. Further, the fluid supplying part is configured such that its tip part is removably inserted into the first path, and supplies fluid to enlarge the dimension of the second blocking part in a state in which the tip part is arranged inside the first path. The liquid supplying part supplies liquid into a space between the first blocking part and the second blocking part.

Further, an ultrasound diagnosis apparatus according to one example of embodiments includes an ultrasound medical apparatus of an embodiment and an image generating part. The image generating part generates image data by processing signals based on echoes received by the ultrasound transducer.

### First Embodiment

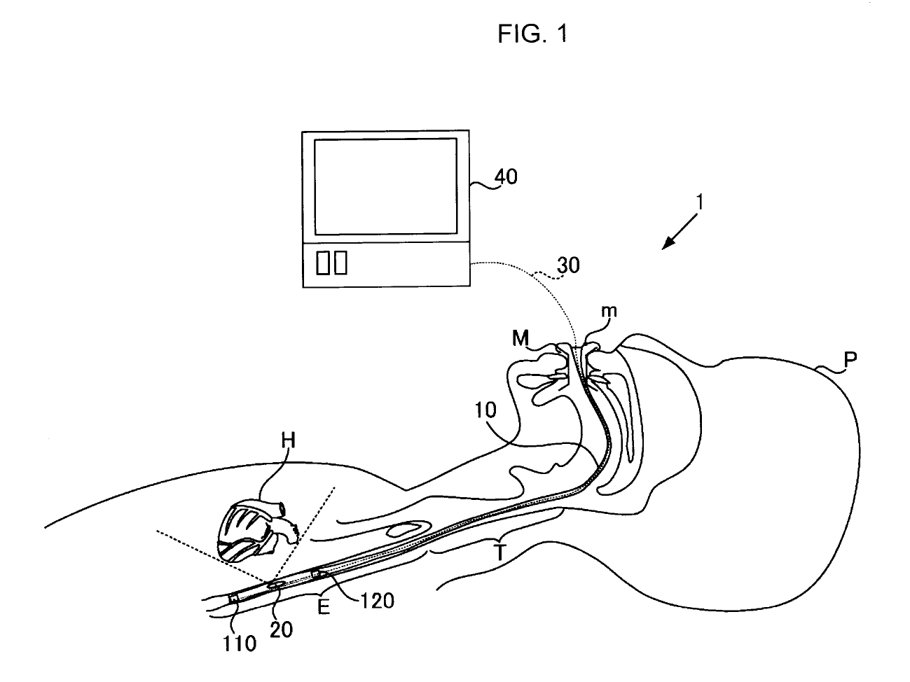

An ultrasound medical apparatus pertaining to the first embodiment as well as an ultrasound diagnosis apparatus 1 including the ultrasound medical apparatus will be described with reference to FIGS. 1 to 9F. The observation objects in this embodiment are the heart and the surrounding vascular system H. It should be noted that the observation objects may be other organs such as a tumor occurred in the esophagus, large intestine, pancreas, spleen, gallbladder, etc., without being limited to the heart.

FIG. 1 illustrates a usage pattern of the ultrasound diagnosis apparatus 1. The ultrasound diagnosis apparatus 1 is used for observing, for example, the heart and surrounding vascular system H from esophagus E, that is, for Transesophageal Echocardiography. The ultrasound diagnosis apparatus 1 comprises: a part (a probe part) to be inserted into the lumen from an insertion opening (a mouth in the present example; another example is the anus etc.) of a subject P; and a part arranged outside the subject P (an external device 40). FIG. 1 illustrates a state in which the tip of the probe part has passed through a throat T and been inserted into an esophagus E.

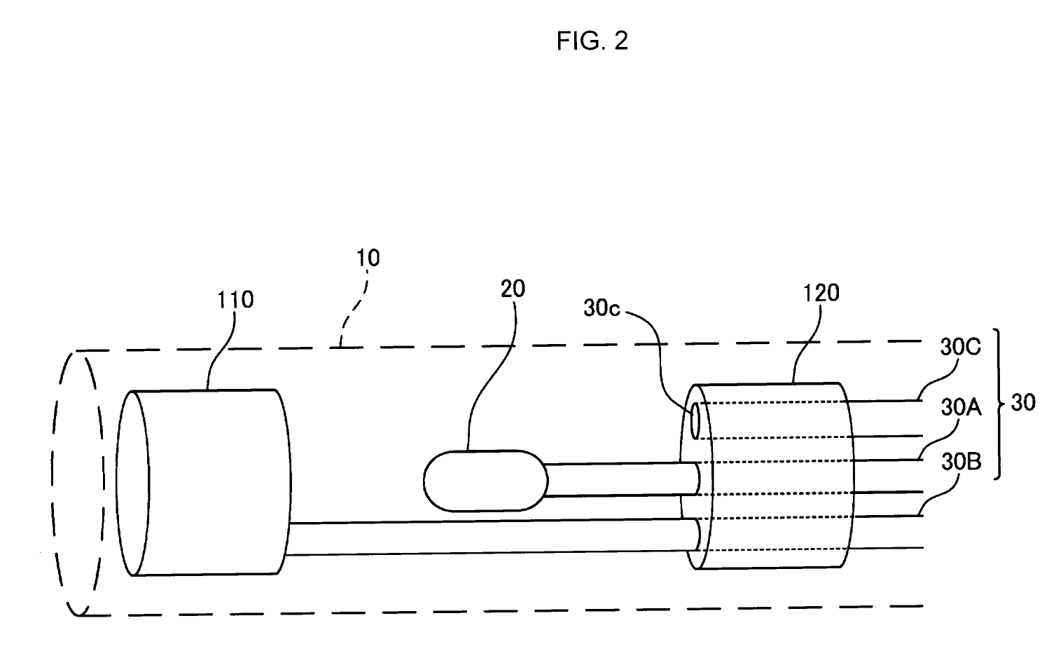

As shown in FIG. 2, the probe part comprises a guiding hollow tube 10, a main body part 20, a front blocking part 110, a rear blocking part 120 and a cable part 30.

### (Cable Part 30)

In this embodiment, the cable part 30 is configured to include three cable parts 30A to 30C.

With regard to the first cable part 30A, the tip side thereof is connected to the main body part 20 while the base end side is connected to the external device 40. The first cable part 30A functions as a signal line for transmitting signals between the main body part 20 and the external device 40.

With regard to the second cable part 30B, the tip side thereof is connected to the front blocking part 110 as well as the rear blocking part 120, while the base end side is connected to the external device 40. In this embodiment, the tip side of the second cable part 30B penetrates the rear blocking part 120 and extends to the front blocking part 110. The second cable part 30B functions as a member for the external device 40 to independently control the front blocking part 110 as well as the rear blocking part 120. This member is configured according to the front blocking part 110 and the rear blocking part 120. For example, with details to be provided later, if configured such that the outer dimensions of the front blocking part 110 and the rear blocking part 120 vary when receiving a supply of fluid, a tubular member that enables the path of the fluid is used as the second cable part 30B. Alternatively, if configured such that the outer dimensions of these are mechanically altered, a signal line for transmitting signals to operate an actuator for this is used as the second cable part 30B. It should be noted that if changes in the outer dimensions of the front blocking part 110 and the rear blocking part 120 are realized by different methods, different members according to these methods are included in the second cable part 30B.

With regard to the third cable part 30C, the tip side thereof is connected to the rear blocking part 120, while the base end side is connected to the external device 40. The third cable part 30C functions such that the external device 40 supplies liquid to the space between the front blocking part 110 and the rear blocking part 120. In this embodiment, the third cable part 30C is a tubular member. This tubular member penetrates the rear blocking part 120, while an opening 30c of the tip thereof is arranged on the front side face of the rear blocking part 120 (that is, the face on the front blocking part 110 side) (ref. FIG. 2). It should be noted that as long as the tip of this tubular member is exposed in the space between the front blocking part 110 and the rear blocking part 120, the position is not limited to the front face of the rear blocking part 120.

In this embodiment, three cable parts 30A to 30C are provided; however, the configuration is not limited to this. For example, the functions of the three cable parts 30A to 30C may be taken on by a cable part 30 that is composed of an arbitrary number (one or a plurality) of cable parts. As a specific example, if the same path is used to supply fluid to the front blocking part 110 as well as the rear blocking part 120 and supply liquid to the space therebetween, by commonly using the second cable part 30B and the third cable part 30C, the number of members included in the cable part 30 is reduced. If the cable part 30 comprises a plurality of cable parts, the cable part 30 may be configured by integrally bundling these. If the cable part 30 is configured so as to realize other functions (for example, a liquid circulation function to be described later), a member for this is provided in the cable part 30. It is possible to configure the cable part 30 such that at least one function (for example, a function that is realized by signal transmission) contributed by the cable part 30 may be conducted by wireless communication.

The cable part 30 in this embodiment has flexible properties. That is, the cable part 30 is formed in a so-called string form. Therefore, when the main body part 20 etc. is placed in the lumen (esophagus E) of the subject P, the cable part 30 is arranged following the shape of the lumen (ref. FIG. 1).

In this embodiment, it is possible to configure the cable part 30 so as to vary the flexible properties when at least a part thereof is twisted. Such configuration is described using the first cable part 30A as an example. It should be noted that the same applies to the second cable part 30B and the third cable part 30C. Furthermore, if the cable part 30, in which the first to third cable parts 30A to 30C are bundled, is adopted, the same configuration may be applied with respect to the cable part 30. Moreover, if configured such that two or more among the main body part 20, the front blocking part 110 and the rear blocking part 120 are configured so as to be substantially and integrally movable (for example, if the portion of the first cable part 30A between the main body part 20 and the rear blocking part 120 does not have substantially flexible properties, or if the portion of the third cable part 30C between the front blocking part 110 and the rear blocking part 120 does not have substantially flexible properties), it is not necessary to apply the configuration of changing flexible properties to all of the cable parts 30A to 30C.

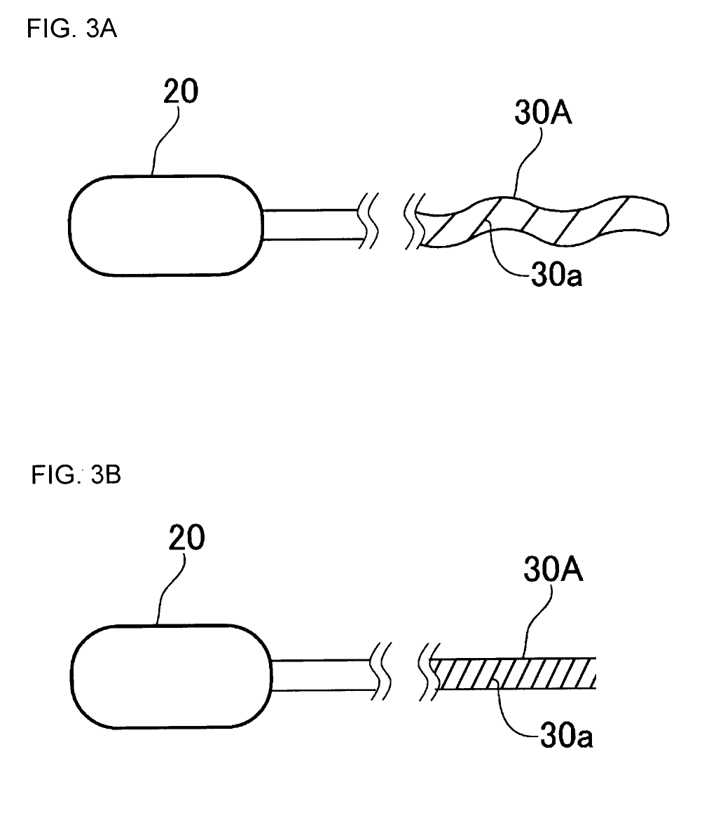

As shown in FIG. 3A, a groove 30a is provided on the outer circumferential face of the first cable part 30A. The flexible properties decline as a result of being twisted along the groove 30a by twisting the first cable part 30A in a predetermined direction (that is, the first cable part 30A enters a state like twisted paper form, ref. FIG. 3B). As described, by pushing in the first cable part 30A with reduced flexible properties in such twisted paper state, the main body part 20 may be forwarded depthwise (in the direction toward the stomach in the present example) of the lumen. It should be noted that by pulling the first cable part 30A, the main body part 20 may be moved in the reverse direction (in the direction toward the mouth in the present example). Such movement in the reverse direction may be conducted in a state having flexible properties by twisting in the reverse direction against the twisting direction with respect to the first cable part 30A.

A string-like member configured for moving the main body part 20, the front blocking part 110 and the rear blocking part 120 may be provided separately from the cable part 30. One end of this string-like member is connected to the rear end of the main body part 20 etc. Furthermore, the string-like member has a structure in which, for example, when twisted, the flexible properties change as in the above configuration.

As a result of employing such a configuration as described above, an operator may move the main body part 20 etc. by pushing or pulling the cable part 30 (or the string-like member). Thereby, the main body part 20 may be moved without directly contacting the wall face of the lumen, enabling the prevention of the wall face from being damaged.

### (Guiding Hollow Tube 10)

The guiding hollow tube 10 is a tubular member covering a series of the main body part 20, the front blocking part 110, the rear blocking part 120 and the cable part 30 of the probe part, and is orally inserted into the lumen of the subject P. The guiding hollow tube 10 is a member for making the operation of guiding the probe part into the lumen easier and is gradually pulled out from the insertion opening in order to successively place the front blocking part 110, the main body part 20 and the rear blocking part 120 in the lumen. Once the probe part is completely placed in the lumen, the entire guiding hollow tube 10 is pulled out from the insertion opening. As an example of a method to make the operation easier, a mouth piece M is attached to the insertion opening of the subject P, enabling the guiding hollow tube 10 to be inserted inside the body via this mouth piece M.

The guiding hollow tube 10 is of a predetermined length and is a member with a hollow having opening parts formed on both ends thereof. A slit may be formed in the longitudinal direction of the side face in order to make the operation of covering a series of the main body part 20, the front blocking part 110 and the rear blocking part 120 of the probe part easier. The length of guiding hollow tube 10 is determined according to an observation object. If the heart and surrounding vascular system H are to be observed, the main body part 20 is arranged at an arbitrary position within the esophagus E. Therefore, the guiding hollow tube 10 to be used is of at least a length to reach from the insertion opening of the subject P to the proximity of the terminal end of esophagus E (gastric cardia). Moreover, the length of the guiding hollow tube 10 may be estimated based on factors related to the length of a gastrointestinal tract, such as body shape, age and/or gender, and a plurality of guiding hollow tubes of different lengths may selectively be adopted. Furthermore, the guiding hollow tube 10 may be formed from a stretchable material.

The guiding hollow tube 10 may be provided with a marker m indicating the distance from the tip (an end part on the insertion side into the body) of the guiding hollow tube 10 may be provided. The marker m is, for example, composed of a predetermined graphic form or a scale. Using the marker m as a reference, the operator is able to assess how far the guiding hollow tube 10 has been inserted into the lumen of the subject P (that is, the location of the main body part 20 in the lumen of the subject P). The location to provide the marker m is determined, for example, based on a standard length from a mouth to esophagus E. It is also possible to prepare a plurality of the guiding hollow tubes 10 in which the makers m are provided at different locations according to factors related to lengths of gastrointestinal tracts, such as body shape, age, and/or gender. Furthermore, if the same kind of examination was conducted on the concerned subject in the past, it is possible to configure to record the insertion length of the guiding hollow tube 10 in the past examination, and conduct a new examination using the guiding hollow tube 10 provided with the marker m at a location according to the recorded insertion length. It should be noted that similar kinds of markers may also be provided in the cable part 30.

### (Main Body Part 20)

The main body part 20 has, for example, a capsule shape appearance so as to easily pass through a throat T of the subject P. At least part (ultrasound wave transmitting and receiving window) of the outer shell of the main body part 20 is formed from a material through which ultrasound waves are permeated (that is, a material whereby ultrasound waves are not substantially reflected/attenuated). A configuration for transmitting and receiving ultrasound waves (described later) is housed in the main body part 20.

The main body part 20 transmits ultrasound waves toward the heart and surrounding vascular system H in a state arranged inside the esophagus E and receives reflected waves from the heart and surrounding vascular system H as echo signals. In the present specifications, the transmission of ultrasound waves and reception of reflected waves are sometimes collectively referred to as “transmitting and receiving ultrasound waves.” The main body part 20 transmits echo signals to the external device 40 via the cable part 30 (the first cable part 30A). The external device 40 processes the echo signals received from the main body part 20 to create and display ultrasound images. The internal configuration of the main body part 20 is described later.

### (Front Blocking Part 110/Rear Blocking Part 120)

The front blocking part 110 and the rear blocking part 120 are respectively configured such that the outer dimensions change. The outer dimensions of the front blocking part 110 and the rear blocking part 120 change at least in the radial directions (that is, in the direction perpendicular to the arrangement direction of the main body part 20, the front blocking part 110 and the rear blocking part 120, in other words, in the radial direction of the lumen).

When the front blocking part 110 and the rear blocking part 120 are being inserted into the lumen and when these are being pulled out from the lumen, the outer dimensions of these are set to “a small diameter state.” That is, the front blocking part 110 and the rear blocking part 120 are inserted into the lumen and are pulled out from the lumen in a state of reduced outer dimensions. Thereby, the insertion operation into the lumen and the removing operation from the lumen may easily be conducted.

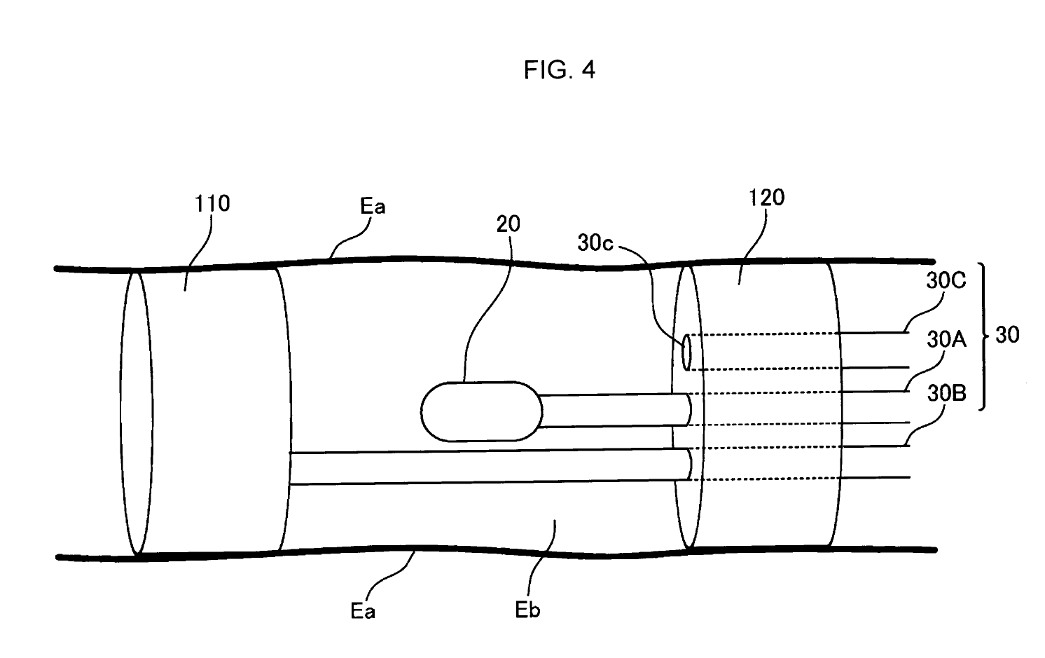

In contrast, when imaging using ultrasound waves, the outer dimensions of the front blocking part 110 and the rear blocking part 120 are set to “a state with a large diameter.” That is, ultrasound waves are transmitted and received by the main body part 20 when the outer dimensions of the front blocking part 110 and the rear blocking part 120 are in an enlarged state. When the outer dimensions of the front blocking part 110 and the rear blocking part 120 are in large diameter states, they substantially block the lumen. “Substantially blocked” means that liquid supplied into the space therebetween does not leak at all between the front blocking part 110 etc. and the wall face of the lumen, or means that the leak is limited and does not affect the ultrasound examination. FIG. 4 illustrates a state in which the front blocking part 110 and the rear blocking part 120 depicted in FIG. 2 are substantially blocking the lumen. The symbol Ea indicates the internal wall of the esophagus E.

While in an inserted state in the lumen, the front blocking part 110 is arranged on the opposite side from the insertion opening into the subject P with respect to the main body part 20. That is, the front blocking part 110 is arranged at a deeper position in the lumen than the main body part 20. Furthermore, the outer circumferential face of the front blocking part 110 with enlarged outer dimension abuts the internal wall Ea of the esophagus E. In contrast, the rear blocking part 120 is arranged at a location closer to the insertion opening than the main body part 20. Furthermore, the outer circumferential face of the rear side blocking part 120 with enlarged outer dimension abuts the internal wall Ea of the esophagus E. As described, by enlarging the outer dimensions of the front blocking part 110 and the rear blocking part 120, the space Eb therebetween (that is, the space surrounded by the front blocking part 110, the rear blocking part 120 and the internal wall Ea) is substantially closed from the surroundings at least with regard to the movement of liquid, while the area surrounding the main body part 20 is filled with liquid. The main body part 20 transmits and receives ultrasound waves in the direction of the heart and the surrounding vascular system H through the space Eb.

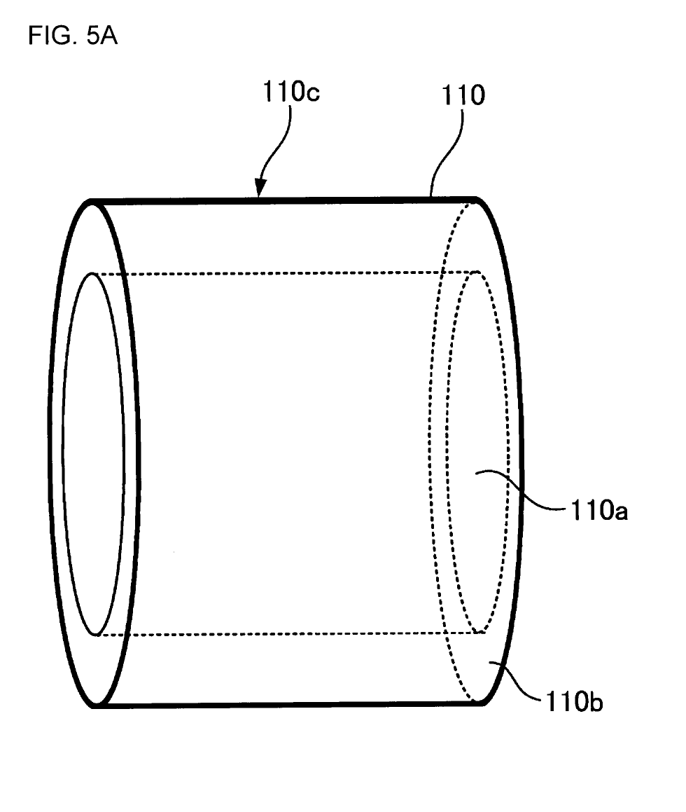

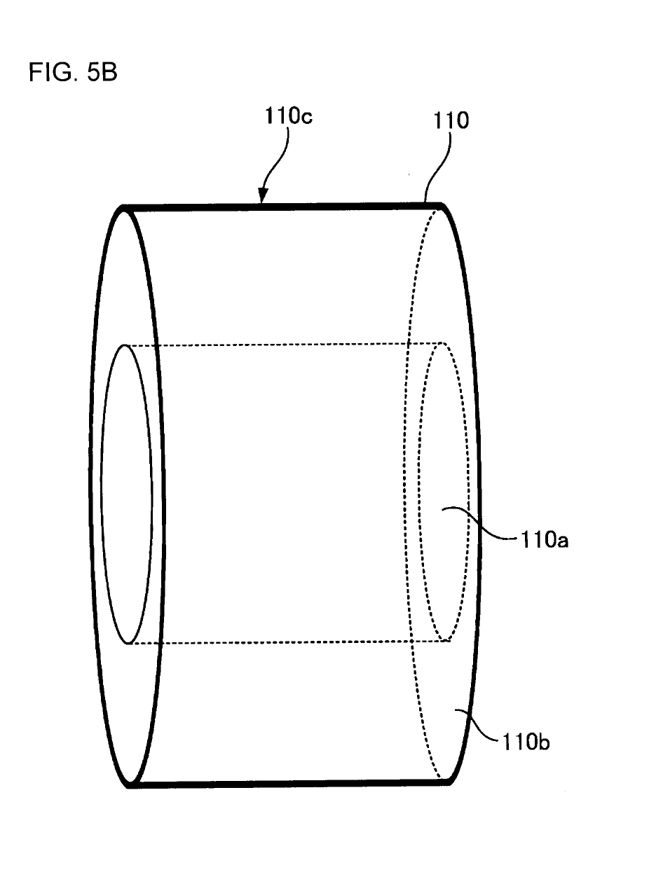

A configuration example of the front blocking part 110 and the rear blocking part 120 that functions as above is described. FIG. 5A and FIG. 5B represent outline configurations of the front blocking part 110. It should be noted that parts not related to the enlargement of the outer dimensions are not illustrated. Furthermore, the rear blocking part 120 may also have the same configuration.

The front blocking part 110 depicted in FIG. 5A comprises a base part 110a and an inflating part 110b. The base part 110a is formed, for example, in a columnar shape. Furthermore, the inflating part 110b is formed, for example, in a cylindrical shape, with the inner circumferential face thereof attached to the outer circumferential face of the base part 110a. Thereby, the inflating part 110b is held by the base part 110a. The inflating part 110b is configured such that the outer dimensions are enlarged by inflation as a result of receiving a supply of fluid from outside. Eventually, the outer dimension of the front blocking part 110 expands in the radial dimension of the lumen (ref. FIG. 5B). In contrast, the inflating part 110b shrinks as a result of discharging fluid that has been filled therein. As described, in the present example, changes in the outer dimensions are realized as changes in the distance from the base part 110a to the outer circumferential face 110c of the inflating part 110b. The outer circumferential face 110c of the inflating part 110b is caused to abut the internal wall Ea of the esophagus E when the outer dimension is in an enlarged state. Therefore, the outer circumferential face 110c is sometimes referred to as an “abutting part.”

The inflating part 110b may be configured as a so-called balloon. Alternatively, the inflating part 110b may be composed from a resilient material. Further alternatively, the inflating part 110b may be configured as a foldable type. That is, the inflating part 110b may be configured so as to be inflated by rolling out creases and shrunk by restoring the creases.

The configurations of the front blocking part 110 and the rear blocking part 120 are not limited to the above inflating or shrinking as a result of fluid movement. For example, it is possible to adopt a configuration in which the outer dimensions are mechanically changed. In this case, an actuator operated by external signals and a mechanism whose outer dimension varies based on drive power generated by the actuator are provided in the front blocking part 110 etc. Examples of such a mechanism include a foldable-type arm that can be bent or extended and a diaphragm blade mechanism comprising a plurality of blade-like members capable of changing their overlapped regions.

As previously described, the outer circumferential face 110c of the inflating part 110b of the front blocking part 110 functions as an abutting part that abuts the internal wall Ea of the esophagus E in a state in which the inflating part 110b is inflated. A part including at least part of the outer circumferential face 110c of the inflating part 110b may be composed from a resilient member. For example, a part of the inflating part 110b with a predetermined thickness having the outer circumferential face 110c as its outer rim may be composed from a resilient member. Furthermore, if a balloon is used as the inflating part 110b, the entire inflating part 110b functions as a resilient member by filling the inside thereof with fluid. Moreover, only a part of the outer circumferential face 110c may be resilient. If at least a part of the outer circumferential face 110c is configured to be resilient as in the above, it is possible to prevent generation of a gap between the front blocking part 110 and the internal wall Ea. Consequently, the blocking properties of the space Eb between the front blocking part 110 and the rear blocking part 120 may be improved. Likewise, with regard to the rear blocking part 120, at least a part of the abutting part may be configured to be resilient.

For at least a part of the outer circumferential face 110c of the inflating part 110b, it is possible to use material in which the viscosity increases by adding water. This member is composed from, for example, a material that is turned into a gel-like state by adding water. By adopting such a configuration, the viscosity of the member increases via liquid supplied into the space Eb between the front blocking part 110 and the rear blocking part 120 or by body fluid, enabling the prevention of a gap being generated between the front blocking part 110 and the internal wall Ea. Thereby, the blocking properties of the space Eb may be improved. Likewise, with regard to the rear blocking part 120, it is possible to use material in which the viscosity increases by adding water for at least part of the abutting part.

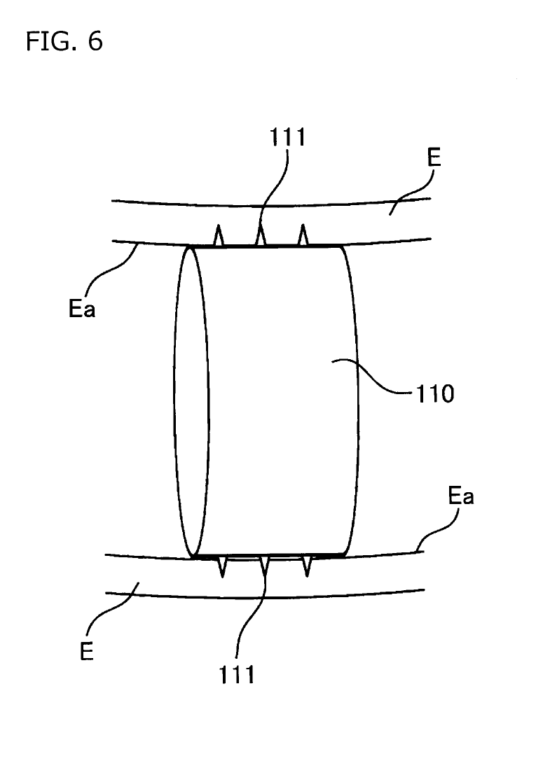

For at least part of the outer circumferential face 110c of the inflating part 110b, a protrusion part protruding toward the internal wall Ea (that is, protruding toward the outside of inflating part 110b in the radial direction thereof) may be provided. An example of the protrusion part is depicted in FIG. 6. A plurality of needle-shaped protrusion parts 111 are provided on the outer circumferential face (abutting part) of the front blocking part 110 pertaining to the present example. When the outer dimensions of the front blocking part 110 are enlarged and the outer circumferential face thereof comes to abut the internal wall Ea of the esophagus E, the needle-shaped protrusion parts 111 stick into the esophagus E. Thereby, the front blocking part 110 is stably secured to the esophagus E. The present example is considered to be effective in the case of monitoring the heart of a subject for a long period of time during lifesaving treatment inside or outside of a hospital or while being transported to a hospital. It should be noted that the protrusion part is not limited to those in a needle shape. For example, the protrusion part may be one whereby friction against the internal wall Ea is increased as a result of increasing the surface area of the abutting part, or may be one with a shape that opposes the movement of the front blocking part 110 caused by the movement (peristalsis) of the internal wall Ea (for example, a shape that is bent toward the stomach side).

### (External Device 40)

The external device 40 at least has a function of controlling the probe part (for example, the main body part 20, the front blocking part 110 and the rear blocking part 120) and a function of creating images based on echo signals (reception data) acquired by the main body part 20. The internal configuration of the external device 40 is described hereinafter.

### (Internal Configuration)

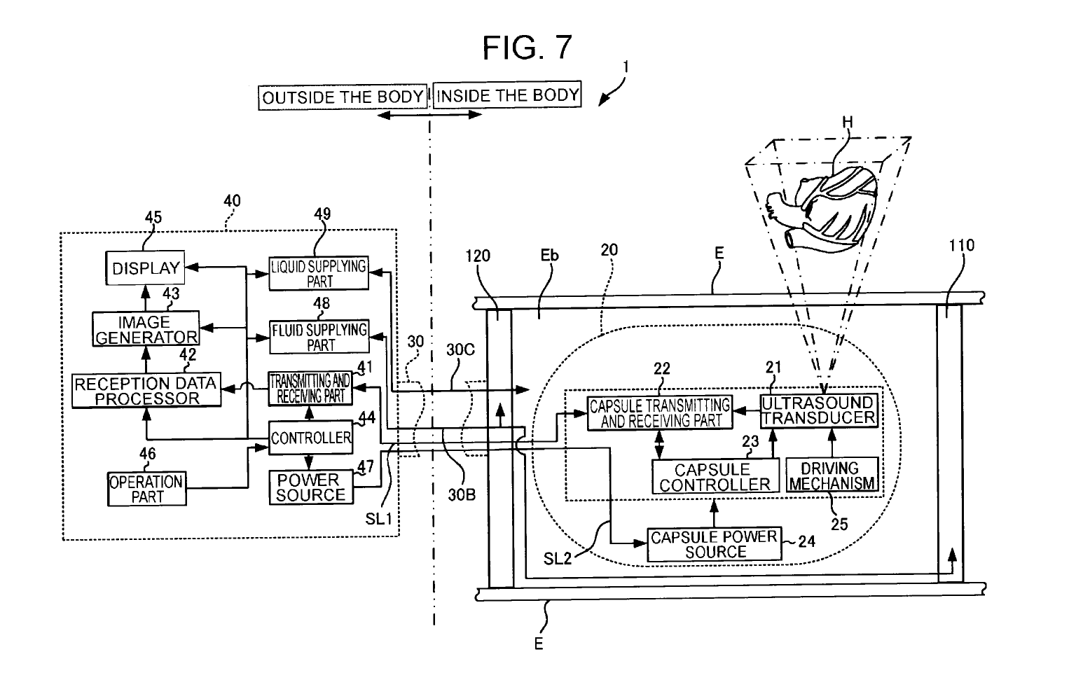

The internal configuration of the ultrasound diagnosis apparatus 1 is described. FIG. 7 is a block diagram depicting one example of the internal configurations of the main body part 20 and the external device 40.

First, the internal configuration of the main body part 20 is described. The main body part 20 is configured comprising an ultrasound transducer 21, a capsule transmitting and receiving part 22, a capsule controller 23, a capsule power source 24 and a driving mechanism 25.

The ultrasound transducer 21 is housed in the main body part 20. The ultrasound transducer 21 transmits ultrasound waves from the radiating face based on drive signals from the capsule controller 23. Furthermore, the ultrasound transducer 21 receives reflected waves from the subject P and transmits echo signals based on the reflected waves to the capsule transmitting and receiving part 22.

As an oscillating element constituting the ultrasound transducer 21, a piezoelectric body or MUT (Micromachining Ultrasound Transducer) element may be used. The MUT element includes a cMUT (Capacitive Micromachining Ultrasound Transducer) or pMUT (Piezoelectric Micromachining Ultrasound Transducer).

In the present embodiment, the ultrasound transducer 21 is, for example, a 2D array type that electrically conducts a scan using a plurality of oscillating elements arranged in a two-dimensional array pattern. According to this 2D array type, it is possible to scan a three-dimensional region in a quadrangular pyramid shape with ultrasound waves (ref. FIG. 7).

The capsule transmitting and receiving part 22 transmits control signals from the external device 40 (a controller 44 to be described later) to the capsule controller 23. The capsule controller 23 transmits drive signals to the ultrasound transducer 21 based on these control signals. Furthermore, the capsule transmitting and receiving part 22 receives echo signals based on the reflected waves received by the ultrasound transducer 21. The capsule transmitting and receiving part 22 transmits these echo signals to the external device 40 (a transmitting and receiving part 41 to be described later). In the present embodiment, transmission of signals between the capsule transmitting and receiving part 22 and the external device 40 is carried out through a signal line SL1 provided in the cable part 30. The signal line SL1 is arranged in the first cable part 30A depicted in FIG. 2.

As a specific example, the capsule controller 23 supplies drive signals to the ultrasound transducer 21 to execute two-dimensional ultrasound scanning. The capsule controller 23 comprises, for example, a clock generator, a transmission delay circuit and a pulsar circuit (not illustrated). The clock generator generates clock signals to determine the transmission timing and/or transmission frequencies of ultrasound signals. The transmission delay circuit performs transmission focusing by applying delay at the time of transmitting ultrasound waves in accordance with the delay time for convergence to converge ultrasound waves to an observation object and the delay time for deflection for transmitting ultrasound waves toward the observation object. The pulsar circuit has the same number of pulsars as individual channels corresponding to oscillating elements. The pulsar circuit generates drive pulses (drive signals) at delayed transmission timing and supplies the drive pulses (drive signals) to oscillating elements that constitute the ultrasound transducer 21. It should be noted that it is also possible to equip the external device 40 with a function as a capsule controller to generate drive pulses (drive signals) of the ultrasound transducer 21 and supply the drive pulses to the ultrasound transducer 21 of the main body part 20. In this case, the power consumption of the main body part may be reduced, and the temperature increase in the lumen due to thermal energy may be reduced.

Furthermore, the capsule transmitting and receiving part 22 conducts delay processing to echo signals received from the ultrasound transducer 21 to convert the analogue echo signals to phased and added digital data (reception data). The capsule transmitting and receiving part 22 comprises, for example, a gain circuit, an A/D converter, a reception delay circuit, and an adder (not illustrated). The gain circuit amplifies (applies a gain to) the echo signals output from the oscillating elements of the ultrasound transducer 21 for each reception channel. The A/D converter converts the amplified echo signals into digital signals. The reception delay circuit provides a necessary delay time to the echo signals that have been converted into digital signals in order to determine the reception directivity. Specifically, the reception delay circuit supplies, to the digital echo signals, the delay time for convergence to converge ultrasound waves from an observation object and the delay time for deflection to set the reception directivity with respect to the observation object. The adder adds the echo signals to which the delay time has been given. By this addition, a reflection component from the direction according to the reception directivity is intensified. That is, the echo signals obtained from the observation object are phased and added by the reception delay circuit and the adder. The capsule transmitting and receiving part 22 outputs the delay processed echo signals (reception data) to the external device 40.

The capsule power source 24 receives a supply of power from the external device 40 (a power source 47 to be described later). The capsule power source 24 distributes the supplied power to the ultrasound transducer 21, the capsule transmitting and receiving part 22 and the capsule controller 23. In the present embodiment, the power supply from the external device 40 is conducted through a signal line SL2 provided in the cable part 30. The signal line SL2 is arranged in the first cable part 30A depicted in FIG. 2.

The driving mechanism 25 moves the movable part of the main body part 20. The movable part includes the ultrasound transducer 21. The movable part may also include a holding part (not illustrated) for holding the ultrasound transducer 21. The driving mechanism 25 is used for altering the position and/or direction to transmit and receive ultrasound waves by the ultrasound transducer 21.

A case of changing the position to transmit and receive ultrasound waves by the ultrasound transducer 21 is described. The driving mechanism 25 functions to move the movable part while in a state of maintaining the orientation of the radiating face of ultrasound waves. In other words, the driving mechanism 25 functions to translate the movable part. For example, the driving mechanism 25 can move the movable part in a direction approaching the front blocking part 110 along with a direction approaching the rear blocking part 120. Thereby, the position of the ultrasound transducer 21 to transmit and receive ultrasound waves may be moved in the arrangement direction of the main body part 20, the front blocking part 110 and the rear blocking part 120 (that is, the longitudinal direction of the lumen (esophagus E)). The direction of translating the movable part by the driving mechanism 25 is not limited to this and may be arbitrary.

Subsequently, a case is described in which the ultrasound transducer 21 changes the direction of transmitting and receiving ultrasound waves. The driving mechanism 25 functions to change the orientation of the radiating face of the ultrasound transducer 21. For example, the driving mechanism 25 can change the orientation of the radiating face of ultrasound waves to the direction perpendicular to the arrangement direction of the front blocking part 110 and the rear blocking part 120 (that is, the radial direction of the lumen (the esophagus E)). This is equivalent to rotation (changes in the rotary angle) of the radiating face of ultrasound waves. Furthermore, the driving mechanism 25 can change the orientation of the radiating face of ultrasound waves to the direction of arrangement of the front blocking part 110 and the rear blocking part 120 (that is, to the longitudinal direction of the lumen (the esophagus E)). This is equivalent to the vertical swing of the radiating face of ultrasound waves (changes in the elevation angle and depression angle). The direction of the deflection of the ultrasound wave radiating face by the driving mechanism 25 is not limited to this and may be arbitrary.

The driving mechanism 25 moves the movable part in response to an operation conducted by the user. The content of this operation is electrically or mechanically delivered to the driving mechanism 25.

An example of the former is described. When the user conducts a desired operation via an operation part 46, the controller 44 controls the transmitting and receiving part 41 to transmit electric signals corresponding to the operation content to the capsule transmitting and receiving part 22 via the signal line SL1. The capsule controller 23 controls the driving mechanism 25 so as to realize the operation content indicated by these electric signals. In this case, the driving mechanism 25 comprises an actuator that is operated and controlled by the capsule controller 23 and a transmission mechanism for delivering driving power generated by the actuator to the movable part. With regard to the actuator, those in which MEMS (Micro Electro Mechanical Systems) technology is used, for example, a capacitive actuator, capacitive micromotor, electromagnetic actuator, piezoelectric actuator, etc. may be used. Likewise, with regard to the transmission mechanism, small size type using MEMS technology may be used. Moreover, it is also possible to mount the actuator and the transmission mechanism integrally on a single substrate.

An example of the latter case is described. The ultrasound diagnosis apparatus 1 comprises an operation equipment exclusively used for moving the radiating face of ultrasound waves. This operation equipment is provided outside the subject P. This operation equipment may be provided as the operation part 46 of the external device 40. The first cable part 30A (ref. FIG. 2) connected to the main body part 20 comprises a mechanism for mechanically delivering the operation content from the operation equipment. This mechanism is, for example, one or more wires, and the operation content is delivered by movement thereof (back and forward movement, rotation, twist, etc.). The driving mechanism 25 interlocks with the movement of the wires and moves the movable part.

Next, the internal configuration of the external device 40 is described. The external device 40 is configured to include the transmitting and receiving part 41, a reception data processor 42, an image generator 43, the controller 44, a display 45, the operation part 46, the power source 47, a fluid supplying part 48, and a liquid supplying part 49.

The transmitting and receiving part 41 transmits control signals from the controller 44 to the capsule transmitting and receiving part 22. Furthermore, the transmitting and receiving part 41 receives echo signals from the capsule transmitting and receiving part 22 and outputs the echo signals to the reception data processor 42.

The reception data processor 42 conducts various kinds of signal processing on the echo signals output from the transmitting and receiving part 41. For example, the reception data processor 42 comprises a B-mode processor. The B-mode processor receives the echo signals from the transmitting and receiving part 41 and converts the amplitude information of the echo signals into a picture. Furthermore, the reception data processor 42 may also comprise a CFM (Color Flow Mapping) processor. The CFM processor converts blood flow information into a picture. Moreover, the reception data processor 42 may comprise a Doppler processor. The Doppler processor extracts Doppler shift frequency components by executing phase detection of the echo signals and executing FFT processing on the extracted components to generate a Doppler frequency distribution that represents blood flow velocity. The reception data processor 42 outputs the echo signals that have been subjected to signal processing to the image generator 43.

The image generator 43 processes the echo signals after the signal processing output from the reception data processor 42 and creates image data (ultrasound image data).

The controller 44 controls actions of each constituent of the ultrasound diagnosis apparatus 1. For example, the controller 44 transmits drive signals for driving the ultrasound transducer 21 to the capsule transmitting and receiving part 22 via the transmitting and receiving part 41 to control the transmission and reception of ultrasound waves. Furthermore, the controller 44 causes the display 45 to display images (ultrasound images) based on image data (ultrasound image data) generated by the image generator 43.

The display 45 comprises a display device such as a liquid crystal display or a CRT. The operation part 46 comprises input devices such as a keyboard, a mouse and/or an operation console. The operator manipulates the operation part 46 for causing the main body part 20 to carry out transmission and reception of ultrasound waves etc.

The power source 47 supplies electricity to the main body part 20 via the signal line SL2. The power source 47 may be configured so as to supply power input from an external power supply to the main body part 20. The external power supply may be a commercial power supply or a battery. Alternatively, the power source 47 may be configured to include an internal power supply (battery, electric cells, etc.)

It should be noted that electric power supply of the main body part 20 may be provided inside the main body part 20. In this case, it is not necessary to provide a power to the main body part 20 from the external device 40, and the signal line SL2 is not necessarily provided. Moreover, by employing publicly known wireless power supply technology, it is also possible to supply power wirelessly from the external device 40 to the main body part 20. In this case as well, the signal line SL2 is not required. Likewise, using publicly known wireless communication technology, it is possible to wirelessly transmit signals between the external device 40 and the main body part 20. In this case, the signal line SL1 is no longer required. Employing such wireless technologies enables the downsizing of the diameter of the cable part 30.

The fluid supplying part 48 is provided for the case in which one or both of the front blocking part 110 and the rear blocking part 120 includes a balloon (inflating part) that is inflated by a fluid (ref. FIG. 5A and FIG. 5B). The fluid supplying part 48 supplies a fluid to the inflating part through the second cable part 30B. The fluid may be liquid or gas. For example, physiological saline solution is used as a fluid.

The fluid supplying part 48 comprises, for example, storage in which fluid is stored, along with a pump to send the fluid in the storage to the inflating part through a tubular member inside the second cable part 30B. It should be noted that the fluid supplying part 48 may be configured so as to send fluid supplied from outside to the inflating part by the pump. In this case, the fluid supplying part 48 receives a supply of fluid from, for example, an external tank or a fluid supply line (for example, waterworks).

The fluid supplying part 48 may have a function to collect the fluid supplied to the inflating part. In this case, the fluid supplying part 48 comprises a pump to suck the fluid inside the inflating part through a tubular member in the second cable part 30B. This pump may either be integrated or separated with respect to the fluid supply pump. Furthermore, the tubular member may also be either integrated or separated with respect to that for fluid supply. The fluid collected from the inflating part is stored in storage that is either integrated or separated with respect to the storage for fluid supply. Alternatively, the fluid collected from the inflating part is discharged outside.

In the case of having such a fluid collection function, the external device 40 is capable of circulating fluid between the fluid supplying part 48 and the inflating part via the second cable part 30B. As a specific example, the controller 44 may control the fluid supplying part 48 to supply new fluid to the inflating part while collecting the fluid that has been supplied to the inflating part. At this time, it is possible to control such that collected volume and supplied volume of the fluid become substantially equal. Thereby, the inflation state of the inflating part does not substantially change, that is, the outer dimensions of the front blocking part 110 and/or the rear blocking part 120 do/does not substantially change. Therefore, the abutting state (abutting pressure etc.) of the front blocking part 110 and/or the rear blocking part 120 to the internal wall Ea of the esophagus E becomes stable. By providing such a fluid circulation function, it becomes possible to discharge heat energy generated from the ultrasound transducer 21 and/or a circuit inside the main body part 20.

The liquid supplying part 49 supplies liquid into the space Eb between the front blocking part 110 and the rear blocking part 120. As for the liquid, for example, degassed physiological saline solution is used.

The liquid supplying part 49 comprises, for example, storage in which liquid is stored, along with a pump for sending the liquid inside the storage to the space Eb through a tubular member in the third cable part 30C. It should be noted that the liquid supplying part 49 may also be configured so as to send liquid supplied from outside to the space Eb by the pump. In this case, the liquid supplying part 49 receives a supply of liquid, for example, from an external replenishing tank or a liquid supply line.

The liquid supplying part 49 may have a function to collect the liquid supplied to the space Eb. In this case, the liquid supplying part 49 has a pump to suck the liquid in the space Eb via a tubular member inside the third cable part 30C. This pump may either be integrated or separated with respect to the liquid supply pump. Furthermore, the tubular member may also be either integrated or separated with respect to the member for liquid supply. The liquid collected from the inside of the space Eb is stored in a storage part that is integrated or separated with respect to the storage part for liquid supply. Alternatively, the liquid collected from inside the space Eb is discharged outside.

In the case in which such a liquid collection function is provided, the external device 40 may circulate the liquid between the liquid supplying part 49 and the space Eb via the third cable part 30C. As a specific example, the controller 44 may control the liquid supplying part 49 to supply new fluid to the space Eb while collecting the fluid that has been supplied to the space Eb. For example, if the space Eb is filled with liquid, it is possible to control so as to substantially equalize the collected volume and the supplied volume. Thereby, it becomes possible to stabilize the pressure applied by liquid inside the space Eb to the front blocking part 110 and/or the rear blocking part 120. By providing such a liquid circulation function, it becomes possible to discharge heat energy generated from the ultrasound transducer 21 or the circuit inside the main body part 20. Furthermore, liquid with a large specific heat may be used to reduce the speed at which temperature rises in the lumen due to this heat energy.

A mode of controlling the fluid supplying part 48 and/or the liquid supplying part 49 may be either automatic control by the controller 44 or manual control by the operator.

It should be noted that it is not necessary to install all of the abovementioned functions of the external device 40 in a single device such as that depicted in FIG. 1. For example, the external device 40 may be configured so as to execute processes up to image data generation by the image generator 43. As a configuration example in this case, the controller 44 may transmit, via a communication line, generated image data to a remote device (for example, a server in a hospital where a doctor is present, or a server in a hospital which is a transporting destination in case of emergency). A doctor in the medical institution which has received the image data may observe ultrasound images based on the image data received from the external device 40. As another configuration example, the controller 44 may record image data generated by the image generator 43 in a recording medium. This recording medium is handed to the destination hospital for emergency and attached to a display device (a computer, an ultrasound diagnosis apparatus, etc.). Thereby, a doctor at the hospital which is an emergency destination may observe ultrasound images based on the image data acquired by the ultrasound diagnosis apparatus 1. Furthermore, by transmitting echo signals received by the external device 40 to a general ultrasound diagnosis apparatus, a doctor at the medical institution may use it in the same way as an extracorporeal ultrasound probe, enabling multiplex ultrasound image diagnosis. An operation part of the external device 40 may also be equipped in a standard ultrasound diagnosis apparatus.

### (Application Modes)

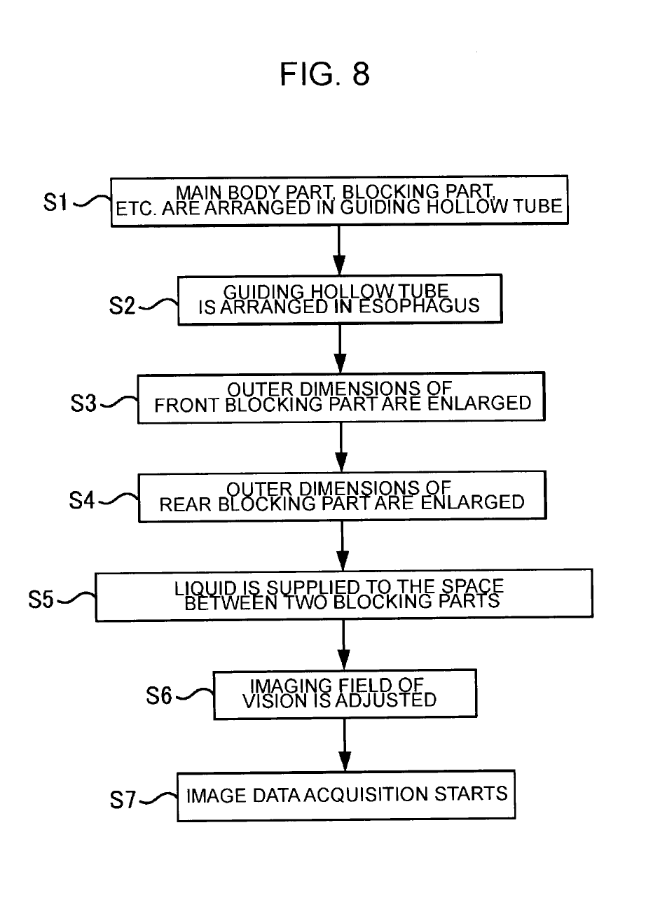

Application modes of ultrasound diagnosis apparatus 1 pertaining to the embodiment are described. One example of the application mode of the ultrasound diagnosis apparatus 1 is depicted in FIG. 8.

(S1: Main Body Part, Blocking Part, Etc. Are Arranged in Guiding Hollow Tube)

First, the user arranges a series of the main body part 20, the front blocking part 110, the rear blocking part 120 and the cable part 30 in the guiding hollow tube 10. The main body part 20, the front blocking part 110 and the rear blocking part 120 are integrated by the cable 30 (ref. FIG. 2).

### (S2: Guiding Hollow Tube is Arranged in Esophagus)

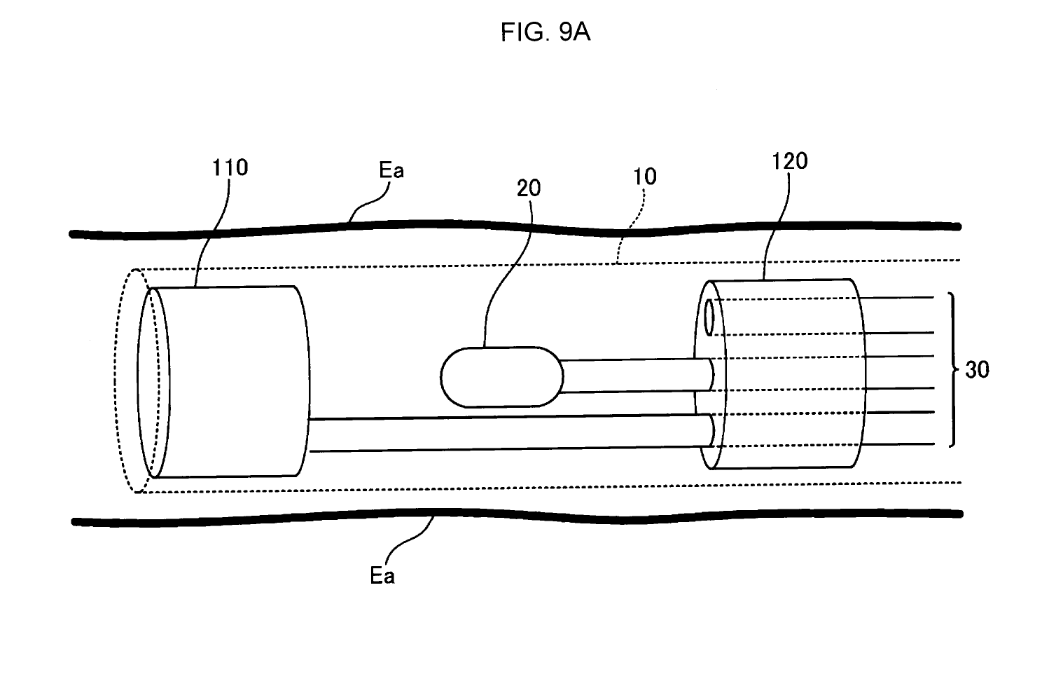

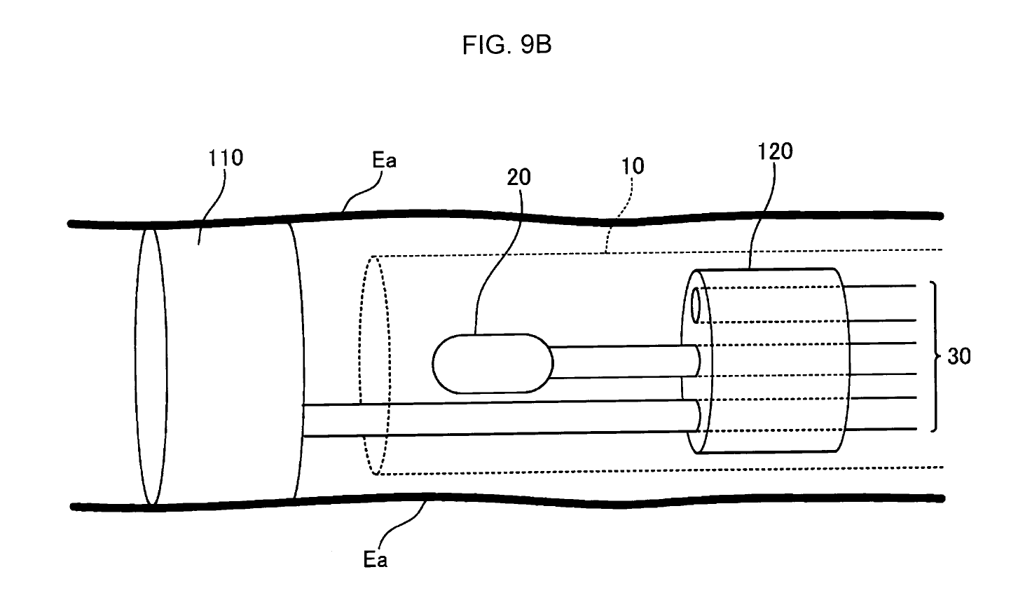

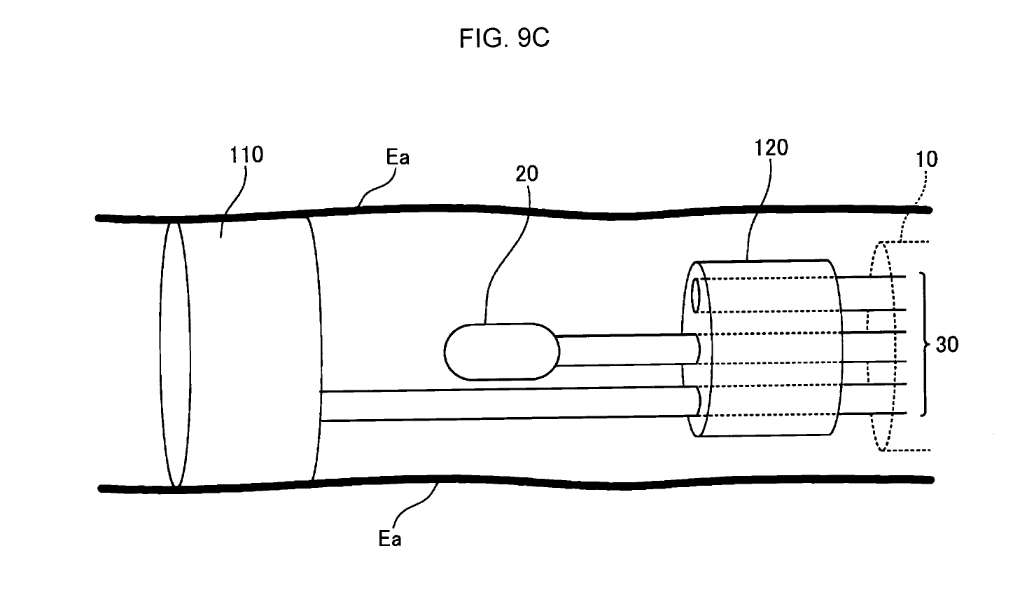

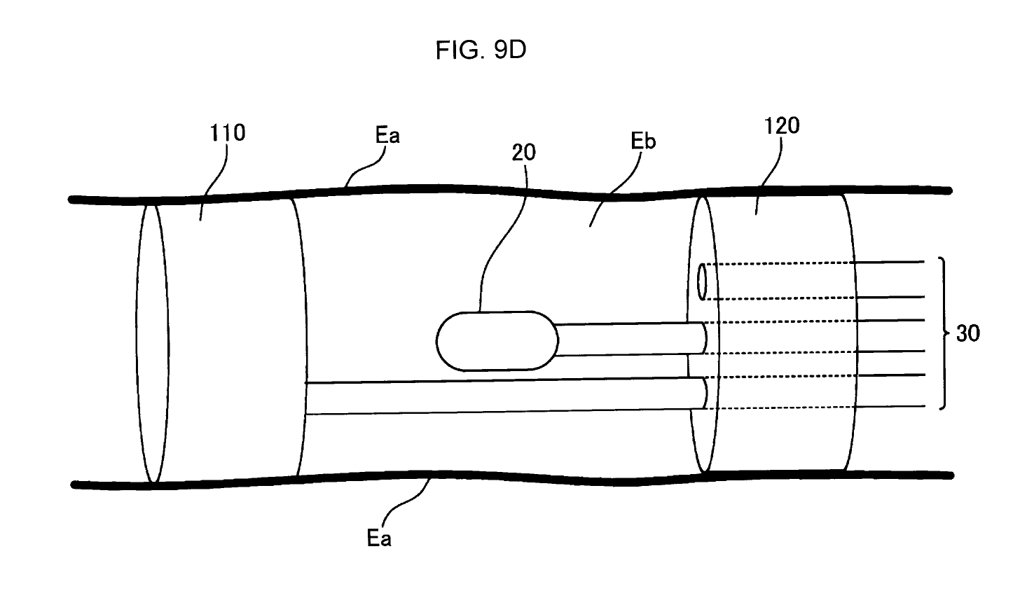

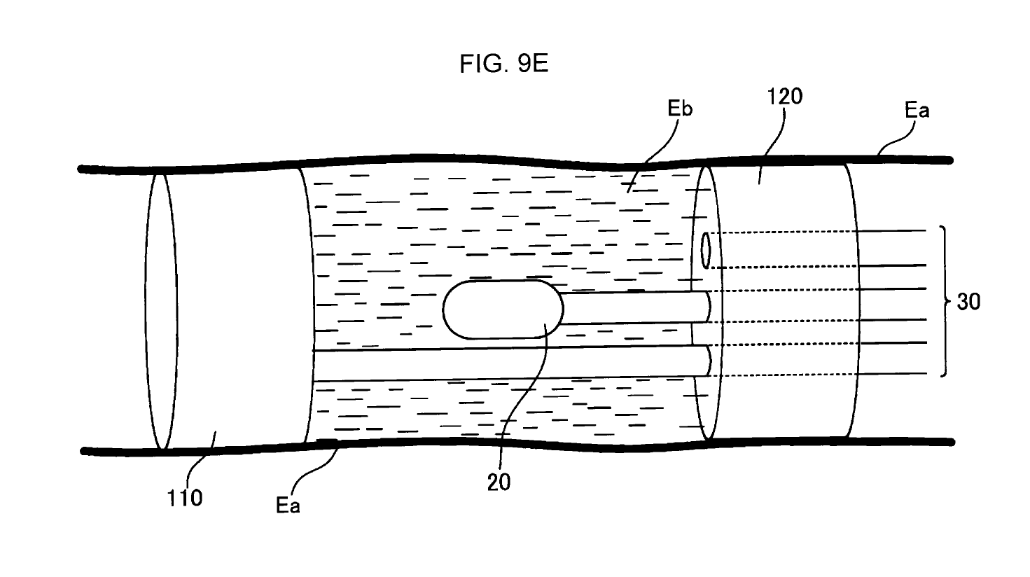

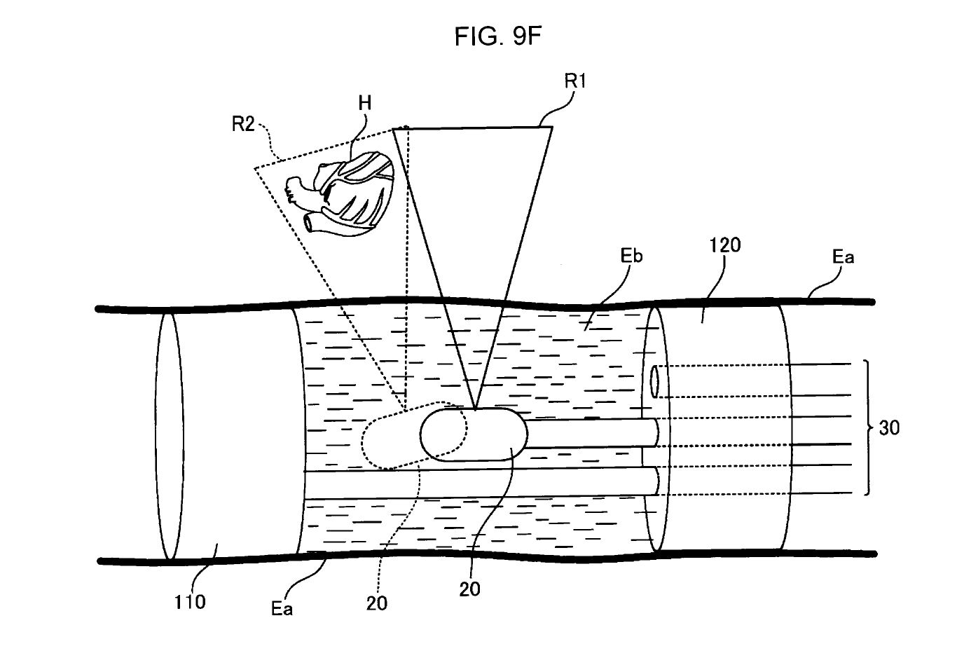

Next, from the insertion opening of the subject P, the user inserts the guiding hollow tube 10, within which the main body part 20 etc. has been arranged in Step S1, so as to be arranged in the esophagus E. For this operation, marker m provided in guiding hollow tube 10 is used as a reference. The user arranges the front blocking part 110 at a desired location by adjusting the location of the tip of the guiding hollow tube 10. This state is depicted in FIG. 9A.

### (S3: Outer Dimensions of Front Blocking Part are Enlarged)

Once the guiding hollow tube 10 is placed at the desired location in Step S2, the user pulls the guiding hollow tube 10 slightly out. The length to be pulled out is approximately to the extent sufficient to expose at least the front blocking part 110 in the esophagus E. Furthermore, the user carries out an operation for enlarging the outer dimensions of the front blocking part 110 via the operation part 46. In correspondence with this operation, the controller 44 controls the fluid supplying part 48 to supply fluid to the inflating part 110b of the front blocking part 110. Consequently, the front blocking part 110 substantially occludes the esophagus E. This state is depicted in FIG. 9B.

It should be noted that the supply volume of the fluid is controlled by the external device 40 or by the user. The case in which control is performed by the external device 40 is described later. On the other hand, if this control is conducted by the user, a means for presenting information indicating the supply level of the fluid to the inflating part 110b may be provided. As an example of this means, a means to monitor the supply pressure of the fluid by the fluid supplying part 48 may be adopted. This supply pressure monitoring means comprises, for example, a pressure sensor for periodically detecting the supply pressure of the fluid. The output from this pressure sensor is input, for example, to the controller 44. The controller 44 causes the display 45 to display pressure values that are periodically input from the pressure sensor. Using the changes in the displayed pressure values as a reference, the user is able to assess the supply level of the fluid. Information that can be captured therefrom may include the presence/absence of contact between the front blocking part 110 and the internal wall Ea of the esophagus E, and/or the level of pressure applied by the front blocking part 110 with respect to the internal wall Ea, etc.

### (S4: Outer Dimensions of Rear Blocking Part are Enlarged)

Once the expansion of the front blocking part 110 is completed in step S3, the user pulls the guiding hollow tube 10 out by a desired amount. The length to be pulled out is approximately to the extent sufficient to expose the main body part 20 and the front blocking part 110 in the esophagus E. FIG. 9C illustrates a state in which the guiding hollow tube 10 has been pulled out to the extent sufficient to expose the rear blocking part 120. It should be noted that at this stage, the guiding hollow tube 10 may be completely pulled out (ref. FIG. 9D). By appropriately changing the flexible properties of the cable part 30 and adjusting the location of the main body part 20 and/or the rear blocking part 120 in the esophagus E, these are placed at desired locations.

Furthermore, the user carries out an operation for enlarging the outer dimensions of the rear blocking part 120 via the operation part 46. In correspondence with this operation, the controller 44 controls the fluid supplying part 48 to supply the fluid into the inflating part of the rear blocking part 120. Consequently, the rear blocking part 120 substantially occludes the esophagus E. This state is depicted in FIG. 9D. It should be noted that control of the supply volume of the fluid may be the same as Step S3.

### (S5: Liquid is Supplied to the Space Between Two Blocking Parts)

When the rear blocking part 120 substantially blocks the esophagus E in Step S4, the space Eb between the front blocking part 110 and the rear blocking part 120 becomes a substantially closed space. The user carries out an operation for supplying liquid into the space Eb via the operation part 46. In correspondence with this operation, the controller 44 controls the liquid supplying part 49 to supply the liquid into the space Eb. The state of the space Eb filled with the liquid is depicted in FIG. 9E.

Control of supply volume of the liquid is conducted by the external device 40 or by the user as in Step S3. It should be noted that the “adjustment of the transmitting and receiving directions of ultrasound beams” described in the following step S6 may be conducted via this control.

If the supply volume of the liquid is controlled by the external device 40, for example, it is possible to control so as to supply the liquid only with a preliminarily determined amount. This preset amount is determined based on the estimated volume of the space Eb. The estimated volume is calculated, for example, as a product of the distance between the front blocking part 110 and the rear blocking part 120 (a fixed value or a value after adjustment) and the cross-section area of the esophagus E (a standard value or an actual measured value).

Another example of a case in which the external device 40 conducts control is described. In parallel, the controller 44 causes the main body part 20 to transmit and receive ultrasound waves, the external device 40 to generate image data, and the liquid supplying part 49 to supply the liquid. By analyzing the image data generated by the image generator 43, the controller 44 determines whether or not inner body tissues (for example, the heart along with the surrounding vascular system H) are favorably depicted. If it is determined that the tissues are favorably depicted, or after the liquid is supplied only by a predetermined amount from the determination, the controller 44 controls the liquid supplying part 49 to stop the supply of the liquid.

A case in which the supply volume of the liquid is controlled by the user is described. Likewise, as described above, the liquid is supplied while acquiring image data. Furthermore, the controller 44 causes the display 45 to display images in real-time based on the image data successively acquired. By referencing the images being displayed in real-time, the user determines whether or not tissues (for example, the heart along with the surrounding vascular system H) are favorably depicted. After confirming that the tissues are favorably depicted, the user carries out an operation, via operation part 46, for terminating the liquid supply.

### (S6: Imaging Field of Vision is Adjusted)

Once the liquid is supplied to the space Eb in step S5, the user or the controller 44 adjusts the imaging field of vision. If this is conducted by the user, the controller 44 causes the images to be displayed in real-time in the same way as above. While referencing the displayed images, the user causes the movable part of the main body part 20 (including the ultrasound transmitting and receiving part 21) to arbitrarily translate, rotationally move, and/or vertically swing in order to adjust the transmitting and receiving directions of ultrasound beams. This adjustment operation continues until the heart along with the surrounding vascular system H, that is an observation target, becomes depicted.

On the other hand, if adjustment of the imaging field of vision is conducted by the controller 44, while acquiring image data as described above, the controller 44 analyzes the acquired image data to determine whether or not the heart along with the surrounding vascular system H (observation target) is being depicted. If it is determined that the heart along with the surrounding vascular system H is not being depicted, the controller 44 transmits control signals for changing the transmitting and receiving directions of the ultrasound beams to the main body part 20 via the transmitting and receiving part 41. The capsule controller 23 controls the driving mechanism 25 to change the transmitting and receiving directions of the ultrasound beams. By repeating such a process in real-time, a search is made for the transmitting and receiving directions of ultrasound beams, enabling depiction of the heart along with the surrounding vascular system H.

FIG. 9F illustrates the outline of the adjustment operation of the imaging field of vision as described above. The user or the controller 44 moves the position of the main body part 20 from the initial location (indicated by the solid line) to a desired location and orientation (indicated by the dotted line). Consequently, the range in which ultrasound waves are transmitted and received is changed from an initial range R1 to a desired range R2. The desired range R2 includes at least a part of the heart along with the surrounding vascular system H, which is an observation target.

### (S7: Image Data Acquisition Starts)

Once the adjustment of the imaging field of vision in Step S6 is completed, the acquisition of image data of the heart along with the surrounding vascular system H starts. The image data is generated by the previously described process. The acquired image data is, for example, used for display processing. Furthermore, it is possible to provide the acquired image data for storage processing. In this case, the controller 44 stores the image data into a predetermined storage device. The destinations of storing the image data include a storage device internally installed in the external device 40, a storage device connected to the external device 40, a recording medium, and other devices (such as a server or data base on a network) etc. The explanation of this application mode is ended here.

### (Effect)

Effects of the ultrasound diagnosis apparatus 1 and the ultrasound medical apparatus included therein pertaining to the present embodiment are described.

The ultrasound medical apparatus (ultrasound probe) included in the ultrasound diagnosis apparatus 1 includes the main body part 20, the front blocking part 110 (first blocking part), the rear blocking part 120 (second blocking part) and the liquid supplying part 49. The main body part 20 comprises the ultrasound transducer 21 configured to transmit and receive ultrasound waves and is inserted into the lumen (the esophagus E) of the subject P. The front blocking part 110 is inserted into the lumen (the esophagus E) and arranged on the side (stomach side) opposite the insertion opening (mouth) of the subject P with respect to the main body part 20. Furthermore, the front blocking part 110 is configured so as to substantially occlude the lumen (the esophagus E) by changing the outer dimensions thereof. The rear blocking part 120 is inserted into the lumen (the esophagus E) and arranged on the insertion opening (mouth) side with respect to the main body part 20. Furthermore, the rear blocking part 120 is configured so as to substantially occlude the lumen (the esophagus E) by changing the outer dimensions thereof. The liquid supplying part 49 is configured so as to supply liquid into the space Eb between the front blocking part 110 and the rear blocking part 120.

Due to such an ultrasound medical apparatus, because ultrasound waves are transmitted and received in a state in which the ultrasound medical apparatus is located inside the lumen of a subject as in a capsule type ultrasound medical apparatus, burdens on the patient becomes relatively small.

Moreover, by supplying liquid into the space Eb, that is substantially blocked, between the front blocking part 110 and the rear blocking part 120, the liquid may be arranged at least in the space between the ultrasound radiating face (ultrasound transmitting and receiving face) of the ultrasound transducer 21 and the wall face on the side of the observation object (the heart along with the surrounding vascular system H). Consequently, ultrasound waves output from the ultrasound transducer 21 propagate in the liquid, pass through the wall face, and reach the observation object. Reflected waves thereof pass through the wall face, propagate in the liquid, and are received by the ultrasound transducer 21. In such a path of ultrasound waves, the only factor attenuating the ultrasound waves is the living body structure from the wall face of the lumen to the observation object. Consequently, it is possible to achieve the same level of image quality as a TEE probe.

Therefore, according to the ultrasound medical apparatus pertaining to the present embodiment and the ultrasound diagnosis apparatus 1 including the same, high quality images of a desired site inside the body may be acquired with low invasion.

In the present embodiment, either one or both of the front blocking part 110 and the rear blocking part 120 may be configured so as to expand the outer dimensions by inflating upon receipt of fluid supply. If this configuration is adopted, the ultrasound medical apparatus comprises the fluid supplying part 48 configured to supply fluid. Furthermore, either one or both of the front blocking part 110 and the rear blocking part 120 comprises an inflating part (the inflating part 110b, etc.) that enlarges the outer dimensions as a result of inflating upon receipt of the fluid supplied from the fluid supplying part 48. Due to this configuration, the expansion of the outer dimensions of the front blocking part 110 and/or the rear blocking part 120 may be realized with a simple structure. It should be noted that as previously described, the configuration for expanding the outer dimensions of the first blocking part and/or the second blocking part is not limited to this.

In the present embodiment, when the outer dimensions of the front blocking part 110 (the rear blocking part 120) are expanded, a part thereof (the outer circumferential face 110c, etc.) comes to abut the wall face of the lumen (the internal wall Ea). This part corresponds to an abutting part. The abutting part may include a resilient member. Due to this configuration, the front blocking part 110 (the rear blocking part 120) comes to abut the internal wall Ea securely. Consequently, the occlusive properties of the space Eb between the front blocking part 110 and the rear blocking part 120 improve.

In the present embodiment, the abutting part of the front blocking part 110 (the rear blocking part 120) may include a member, the viscosity of which increases by adding water. Due to this configuration, it is possible to prevent the generation of a gap between the front blocking part 110 (the rear blocking part 120) and the internal wall Ea. Consequently, the blocking properties of the space Eb between the front blocking part 110 and the rear blocking part 120 improve.

In the present embodiment, the abutting part of the front blocking part 110 (the rear blocking part 120) may comprise a protrusion part that protrudes toward the wall face (the internal wall Ea) of the lumen. Due to this configuration, the front blocking part 110 (the rear blocking part 120) is securely fixed in the lumen. It should be noted that at least the tip of the protrusion part may be formed in a needle shape.

In the present embodiment, the liquid supplying part 49 may comprise the third cable part 30C such as that depicted in FIG. 2 or FIG. 4. The third cable part 30C penetrates the rear blocking part 120. Furthermore, the opening 30c of the tip side of the third cable part 30C is exposed to the space Eb between the front blocking part 110 and the rear blocking part 120. The third cable part 30C is one example of a “second tubular member.” The liquid supplying part 49 may supply liquid into the space Eb via such a second tubular member.

The liquid supplying part 49 may be configured so as to supply new liquid while collecting the liquid having been supplied to the space Eb. That is, the liquid supplying part 49 may circulate the liquid within the path including the space Eb. Due to this configuration, heat energy generated from the ultrasound transducer 21 and/or a circuit inside the main body part 20 may be discharged. Consequently, it becomes possible to stably manipulate the main body part 20.

The ultrasound medical apparatus pertaining to the present embodiment may comprise the guiding hollow tube 10. The guiding hollow tube 10 is used in order to guide the series of the main body part 20, the front blocking part 110, the rear blocking part 120 and the cable part 30. The marker m indicating the length of a part that has been inserted into the subject P may be provided in the guiding hollow tube 10. Due to this configuration, because the length of a part of the guiding hollow tube 10 inserted into the lumen may be verified by the marker m, it is possible to assess the approximate location of the main body part 20, etc. inside the lumen.

In the present embodiment, the main body part 20 may comprise the driving mechanism 25 for moving a movable part including the ultrasound transducer 21. The driving mechanism 25 may be configured so as to move the movable part in the direction approaching the front blocking part 110 as well as the direction approaching the rear blocking part 120. Furthermore, the driving mechanism 25 may be configured so as to move the movable part in order to change the directions of ultrasound waves transmitted and received by the ultrasound transducer 21. Due to this configuration, the location for transmitting and receiving ultrasound waves and/or the transmitting and receiving directions of ultrasound beams may be changed, enabling the ultrasound waves to be favorably transmitted and received with respect to the observation object. For example, imaging of a desired site of the observation object is possible from a desired direction.

In the present embodiment, the cable part 30 may be connected to the main body part 20, the front blocking part 110 and the rear blocking part 120. The cable part 30 may be flexible. Furthermore, the cable part 30 may comprise one or a plurality of cable parts. In the above embodiment, three cable parts 30A to 30C are provided. The cable part 30 is provided with, for example, a signal line (the first cable part 30A) for transmitting signals between the main body part 20 and the external device 40, a member (the second cable part 30B) for changing each of the outer dimensions of the front blocking part 110 and the rear blocking part 120, a member (the third cable part 30C) for supplying liquid into the space Eb between the front blocking part 110 and the rear blocking part 120 from the liquid supplying part 49. Due to such a configuration, the main body part 20 etc. may be smoothly inserted into the lumen by the flexible cable part 30. Furthermore, it is possible to control the main body part 20 etc. via the cable part 30.

A part of or the entire cable part 30 may be configured such that the flexible properties change when twisted. Due to this configuration, in the event of adjusting the location of main body part 20 etc. inside the lumen, the cable part 30 may be forwarded or pulled back while appropriately altering the flexibility of the cable part 30. Consequently, it becomes possible to facilitate manipulation for arranging the main body part 20 etc. to a desired location inside the lumen.

In addition to an ultrasound medical apparatus such as that mentioned above, the ultrasound diagnosis apparatus 1 pertaining to the present embodiment has a function to generate image data by processing echo signals based on reflected waves received by the ultrasound transducer 21. This image generating function is realized via an “image generating part” including the reception data processor 42 and the image generator 43.

The various configurations mentioned above may be arbitrarily combined. Furthermore, it is possible to apply an arbitrary configuration among the abovementioned configurations to embodiments and/or modified examples to be described later.

### MODIFIED EXAMPLES

Modified examples of the ultrasound medical apparatus and the ultrasound diagnosis apparatus pertaining to the present embodiment are described. In the following modified examples, parts differing from the above embodiment are described.

### Modified Example 1

A function to change the interval between the front blocking part 110 and the rear blocking part 120 may be provided. The configuration for realizing this function is arbitrary. In the following example, a connecting member that connects the front blocking part 110 and the rear blocking part 120 is used.

As a first configuration example, it is possible to use a connecting member of a predetermined length configured such that one end thereof is attachable/detachable with respect to the front blocking part 110 while the other end thereof is attachable/detachable with respect to the rear blocking part 120. In the present example, a plurality of connecting members of different lengths is prepared. Moreover, a connecting member of a desired length is selectively adopted.

The connecting member may be a member that can be deformed or a member that is not substantially deformed. In the case of applying a deformable configuration, the connecting member may be a freely deformable member, a member that is flexible, or a member that has plasticity.

In the present example, an engagement part is respectively provided at both ends of the connecting member. Furthermore, an engagement part is respectively provided on the face of the front blocking part 110 on the side of the rear blocking part 120 as well as on the face of the rear blocking part 120 on the side of the front blocking part 110. The engagement part on one end of the connecting member is connected to the engagement part of the front blocking part 110 and the engagement part on the other end thereof is connected to the engagement part of the rear blocking part 120. The front blocking part 110 and the rear blocking part 120 connected to each other via the connecting member as described are inserted into the lumen together with the main body part 20.

The first configuration example is one example of a configuration to change the interval between the front blocking part 110 and the rear blocking part 120 inserted into the lumen. On the other hand, in the second configuration example, a configuration to change the interval while in the state arranged in the lumen is described. Also in the present example, one end of the connecting member is connected to the front blocking part 110, whereas the other end is connected to the rear blocking part 120. In the present example, the connecting member may be configured so as to be attachable/detachable with respect to the front blocking part 110 and the rear blocking part 120, or may also be fixed to these.

Furthermore, the connecting member comprises a mechanism for altering its own length. This mechanism comprises, for example, a first member that includes a female screw with threads formed on the inner circumferential face, a second member including a male screw with threads formed on the outer circumferential face and engaged with the female screw, and an actuator (pulse motor etc.) to relatively rotate the female screw and the male screw. The end part of the first member is connected to the front blocking part 110 (or the rear blocking part 120), while the end part of the second member is connected to the rear blocking part 120 (or the front blocking part 110). The controller 44 controls the actuator by transmitting control signals through the cable part 30. When the female screw and the male screw are relatively rotated by the actuator, the first member and the second member approach each other or move away from each other. Consequently, the length of the connection part alters and the front blocking part 110 and the rear blocking part 120 become relatively closer to or distant from each other. It should be noted that whether to move the front blocking part 110 and the rear blocking part 120 closer to or away from each other depends on the rotating direction of the actuator controlled by the controller 44.

As exemplified above, the ultrasound medical apparatus and the ultrasound diagnosis apparatus including the same pertaining to the present modified example comprise a mechanism of changing the distance between the front blocking part 110 and the rear blocking part 120.

According to the configuration pertaining to the present modified example, it is possible to arbitrarily adjust the interval between the front blocking part 110 and the rear blocking part 120 in accordance with conditions such as the state of the lumen or individual differences.

### Modified Example 2

In the above embodiment, the front blocking part 110 and the rear blocking part 120 are configured such that their outer dimensions alter. On the other hand, the size (thickness, diameter) of the lumen varies among sites or among individuals. In the present modified example, under such conditions, a configuration to enlarge the front blocking part 110 etc. so as to favorably occlude the lumen is described.

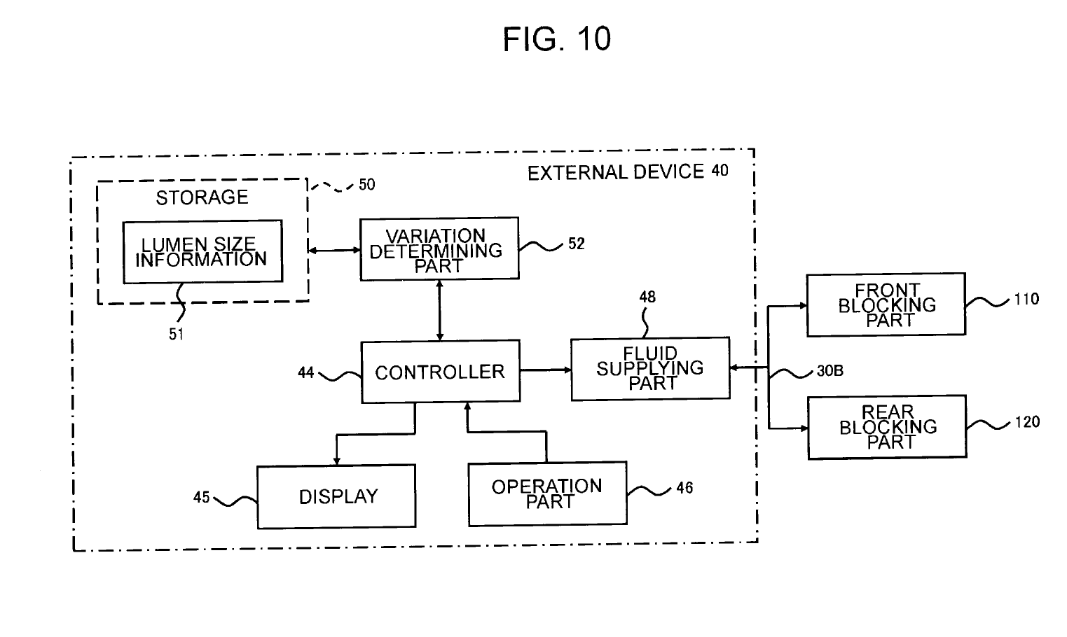

A configuration example pertaining to the present modified example is depicted in FIG. 10. In the present modified example, in addition to the configuration shown in FIG. 7, the external device 40 comprises a storage 50 and a variation determining part 52. The storage 50 stores a lumen size information 51. The lumen size information 51 is generated in advance and stored in the storage 50.

The lumen size information 51 includes information indicating the size of a lumen. Examples of a value indicating the size of the lumen include diameter, radius, circumferential length, cross-section area, etc. Furthermore, this value may be a standard value regarding lumens or may also be a value related to the subject.

The standard value of lumen size may be, for example, a value statistically obtained (mean, mode, median, etc.) from the measured values of lumens of a plurality of subjects. The standard value may be a value stated in a literature such as a treatise. Furthermore, a plurality of values may be recorded as the standard value. In this case, each of the plurality of standard values may be associated with attributes of subjects. Examples of the attributes of subjects include, for example, conditions that affect lumen size and/or conditions that could affect lumen size such as gender, age group, body type (height, weight, etc.), anamnesis, etc. If a plurality of standard values is included in the lumen size information 51, a standard value is selectively applied corresponding to the attributes of a subject who is an object of ultrasound examination. At this time, the attributes of the subject are manually input via the operation part 46, for example. Alternatively, it is possible to configure such that the attributes are obtained by the controller 44 from electronic medical records etc. of the concerned subject. Furthermore, a configuration is also possible in which the display 45 displays a plurality of standard values and a desired standard value is chosen using the operation part 46.

On one hand, if a value related to the concerned subject is included in the lumen size information 51, this value is a measured value resulting from actually measuring the size of the lumen of the subject. This actual measurement value may be associated with, for example, identification information of the subject (patient ID etc.) and stored. Furthermore, an actual measurement value recorded in electronic medical records etc. obtained from an in-hospital information system upon receiving input of the identification information may be used as the lumen size information 51.

The variation determining part 52 determines amounts of change in the outer dimensions (amount of expansion) of the front blocking part 110 and/or the rear blocking part 120 based on the lumen size information 51. Such amounts of change may include information indicating the size of the outer dimensions (diameter, radius, circumferential length, cross-section area, etc.) and/or information indicating the amount of the fluid supplied to the front blocking part 110 etc.

An example of a process to determine the amount of change (variation) is explained. As described, the lumen size information 51 includes a standard value or an actual measurement value of the lumen size. Furthermore, the storage 50 or the variation determining part 52 preliminarily stores information indicating the sizes of the front blocking part 110 and/or the rear blocking part 120 prior to the expansion, that is, information indicating their sizes in a shrunken state (initial size information). This initial size information may include information indicating the volume of the inflating part 110b etc. in the shrunken state and/or information indicating the size of the outer dimensions (diameter, radius, circumferential length, cross-section area, etc.) in the shrunken state. Moreover, in addition to the initial size information or in lieu of the initial size information, the storage 50 or the variation determining part 52 preliminarily stores information (supply amount-variation information) indicating the relationship between supply amounts of fluid to the front blocking part 110 (the rear blocking part 120) and the variation in size of the front blocking part 110 (the rear blocking part 120). It should be noted that the amount of fluid supplied is substantially equal to the change in the volume of the inflating part 110b related to the variation in size.

The variation determining part 52 may determine the amount of fluid supplied to the front blocking part 110 and/or the rear blocking part 120 based on the lumen size information 51 as well as the initial size information and/or supply amount-variation information. As an example of this process, the variation determining part 52 firstly obtains the variation (for example, the variation in diameter) between the size in the shrunken state indicated in the initial size information and the standard size or the actual size indicated in the lumen size information 51. Subsequently, the variation determining part 52 acquires the amount of fluid supply corresponding to the obtained variation in size (diameter etc.) using the supply amount-variation information as a reference. The acquired supply amount of the fluid is used as the outer dimensional variation (enlarged amount) of the front blocking part 110 (the rear blocking part 120).

The variation determining part 52 may execute such a process as described above for each of the front blocking part 110 and the rear blocking part 120. Moreover, it is also possible to determine the variation regarding the rear blocking part 120 (the front blocking part 110) with reference to the variation determined for the front blocking part 110 (the rear blocking part 120). On one hand, if the distance between the front blocking part 110 and the rear blocking part 120 is sufficiently short or in the event the present example is applied to a lumen with minor size variation by sites, the same value of variation may be applied to both the front blocking part 110 and the rear blocking part 120.

The controller 44 controls the fluid supplying part 48 based on the variation determined by the variation determining part 52 to enlarge the outer dimensions of the front blocking part 110 and/or the rear blocking part 120. That is, the controller 44 controls the fluid supplying part 48 so as to supply fluid by the amount of supply determined by the variation determining part 52 to inflate the inflating part 110b of the front blocking part 110 (similar inflating part in the rear blocking part 120).

As exemplified above, the ultrasound medical apparatus and the ultrasound diagnosis apparatus including this pertaining to the present modification example comprise: the variation determining part 52 that determines the outer dimensional variation in at least either one of the front blocking part 110 and the rear blocking part 120 based on the preliminarily generated lumen size information 51; and the controller 44 that changes the outer dimensions of at least one of the front blocking part 110 and the rear blocking part 120 (that expands at least one of these) based on the determined variation. The variation determining part corresponds to the “determining part” and the controller 44 corresponds to the “controller.”

According to the configuration pertaining to this modified example, it is possible to automate processing of substantially occluding the lumen by expanding the front blocking part 110 and/or the rear blocking part 120.

It should be noted that if a standard value is used or if an actual measurement value is acquired via an examination in the past, there is a risk of the possible presence of a gap that cannot be ignored between the value indicated in the lumen size information 51 and the current lumen size. As one example of a method for avoiding such inconvenience, by measuring the lumen size in the current examination, this measured value may be used as the lumen size information 51.

Another method includes the function of monitoring an expansion state while executing the process to expand the front blocking part 110 etc. It should be noted that the expansion state indicates the relationship between the lumen size and the current size of the front blocking part 110 etc. As a specific example, it is possible to configure so as to detect the fact that the front blocking part 110 contacts with the internal wall Ea based on the temporal change in the fluid pressure inside the front blocking part 110. It should be noted that the detection of the fluid pressure is, for example, conducted by a pressure sensor. Alternatively, the temporal change in the fluid pressure may be comprehended by monitoring the operation state (pressure etc. to transfer the fluid) of the pump of the fluid supplying part 48. The expansion state of the front blocking part 110 etc. obtained as described above is presented on the display 45 via the controller 44, for example. The user may give an instruction for a desired process (stop/continuation of the fluid supply) via the operation part 46. Furthermore, it is also possible to adopt a configuration in which the expansion state obtained in real-time is fed back to the control of the fluid supplying part 48 via the controller 44.

### Modified Example 3

In the above embodiment, the front blocking part 110 and the rear blocking part 120 have a function to substantially occlude the lumen. However, because the lumen is a living body organ, there is a risk of liquid leaking from the gap between the front blocking part 110 etc. and the lumen if the lumen cannot be substantially occluded due to individual differences and/or movements (peristaltic movements etc.). In the present modified example, a function to detect whether or not such an inconvenient situation is occurring is described.

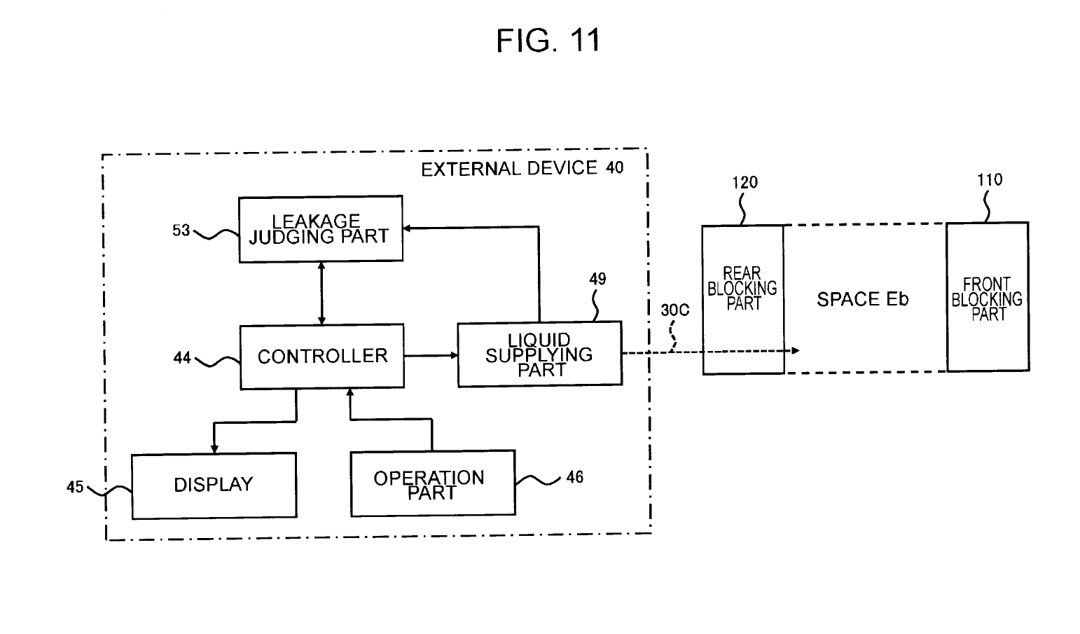

A configuration example pertaining to the present modified example is depicted in FIG. 11. In the present modified example, the external device 40 comprises a leakage judging part 53 in addition to the configuration illustrated in FIG. 7.

The leakage judging part 53 judges whether or not liquid is leaking from the space Eb based on the state of the liquid supply to the space Eb by the liquid supplying part 49. As an example of a process executed by the leakage judging part 53, it is possible to monitor the operation state of the pump (pressure for transferring liquid, etc.) of the liquid supplying part 49 and execute the judging process based on the temporal change in the operation state of the pump. More specifically, until the space Eb is filled with liquid, the transferring pressure by the pump is substantially constant and once having been filled, the transferring pressure changes in accordance with the presence/absence of leakage and the extent thereof. Typically, if there is no leakage, the transferring pressure gradually increases; however, if no leakage exists, the increase in the transferring pressure stops or the transferring pressure decreases at a certain point of time. At this moment, by controlling the supply rate of the liquid (the amount of liquid supplied per unit time) according to the transferring pressure, excessive increases in the pressure of the liquid within the space Eb may be prevented.

The leakage judging part 53 stores information that associates the operation states of the pump with the presence/absence of leakage (or the presence/absence of risk thereof), wherein this information (operation state-leakage information) is preliminarily generated based on the relationship as described above. Subsequently, the leakage judging part 53 judges the presence/absence of leakage based on information indicating the operation state of the pump that has been received from the liquid supplying part 49 and the operation state-leakage information.

It should be noted that the operation state of the liquid supplying part 49 depends on the pressure inside the space Eb or the pressure inside the third cable part 30C as long as there are no factors such as other controls or malfunction of devices, etc. Therefore, the presence/absence of leakage may be judged based on the output from a pressure sensor that detects the pressure inside the space Eb and/or inside the third cable part 30C.

When the leakage judging part 53 judges that the leakage exists, the controller 44 causes predetermined notification information to be output. This notification control includes, for example, control of causing the display 45 to display predetermined visual information (character information, image information, etc.) or control of causing audio outputting part (not illustrated) to output predetermined audio information (alert sound etc.). Similar notification control may also be carried out in the case in which the leakage judging part 53 judges that the leakage does not exist.

As exemplified above, the ultrasound medical apparatus and the ultrasound diagnosis apparatus including the same pertaining to the present modified example comprise: the leakage judging part 53 that judges whether or not liquid is leaking from the space Eb between the front blocking part 110 and the rear blocking part 120 based on the supply state of the liquid by the liquid supplying part 49; and the controller 44 that executes notification control based on the judgment results from the leakage judging part 53. Herein, the leakage judging part 53 corresponds to a “first judging part” and the controller 44 (as well as the display 45, the voice output part, etc.) corresponds to a “first notifying part.”

According to the configuration pertaining to the present modified example, it is possible to automatically detect the leakage of liquid and notify the detection result. Therefore, it may be prevented that an ultrasound examination is carried out under an inappropriate condition in which the liquid inside the space Eb is leaking.

### Modified Example 4

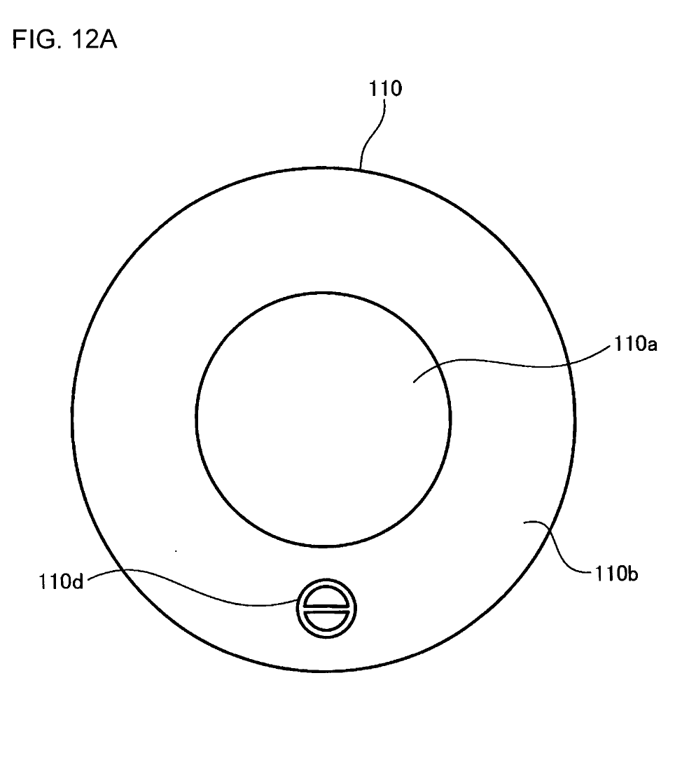

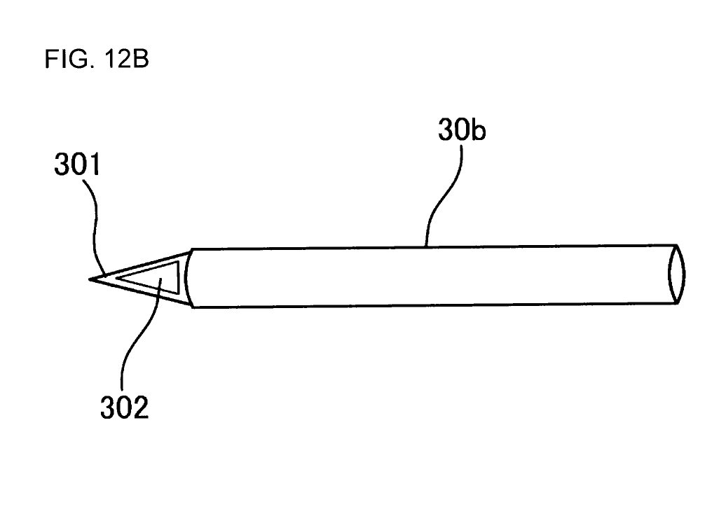

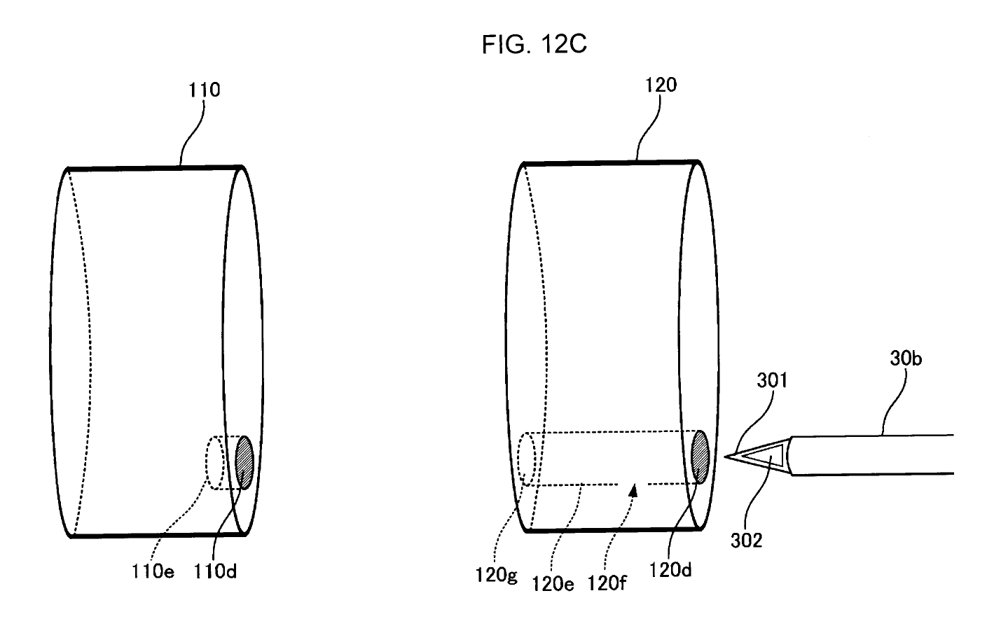

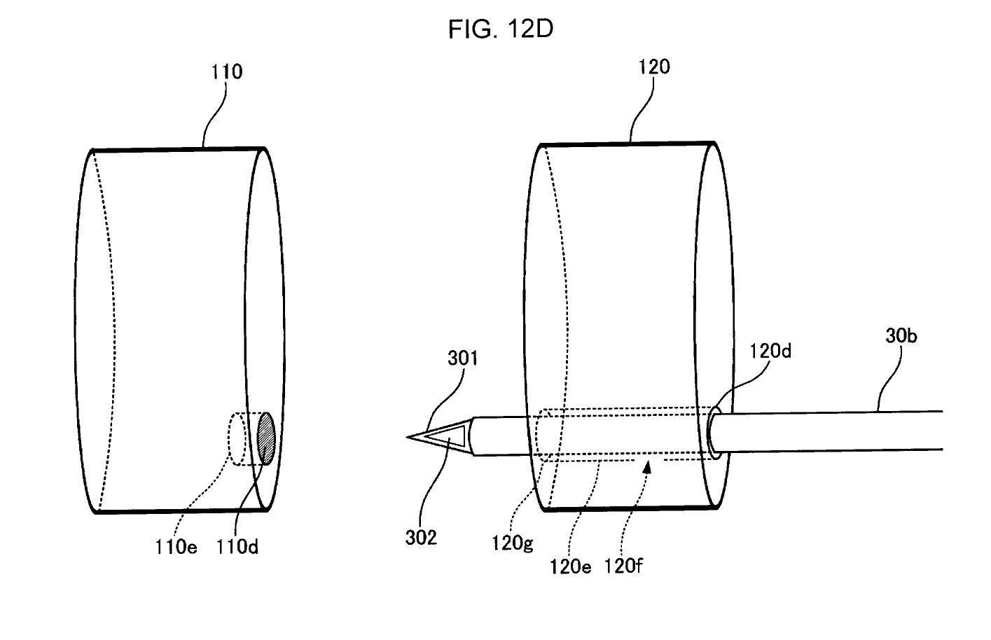

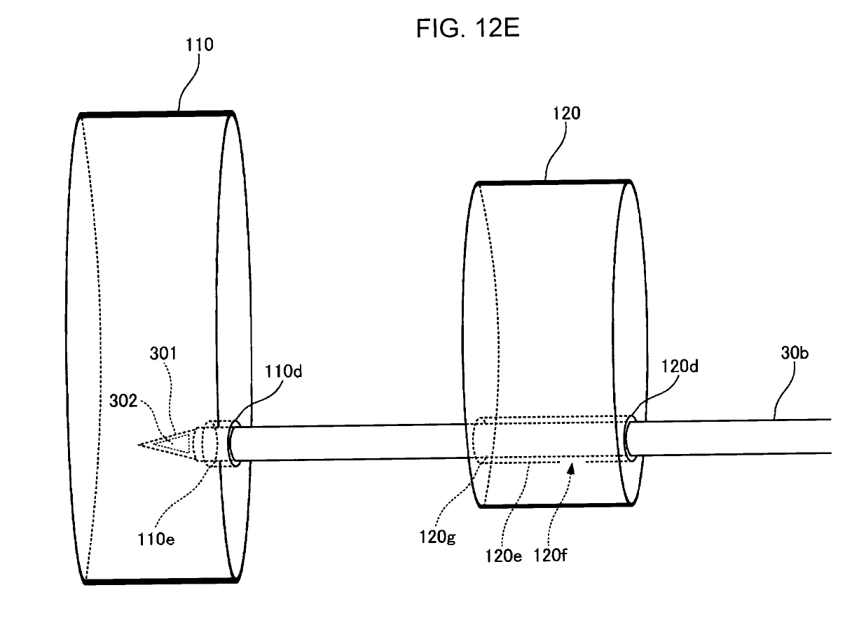

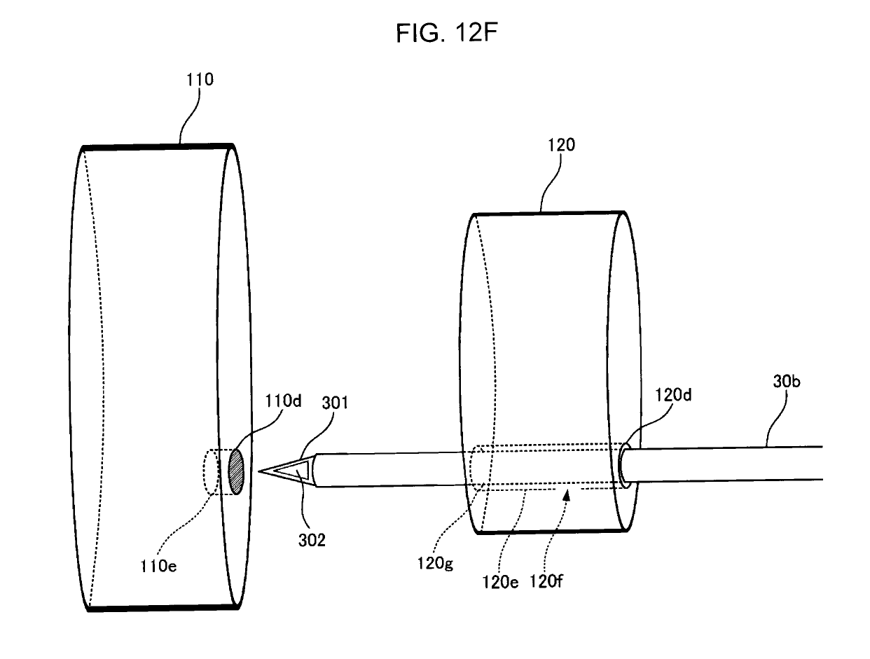

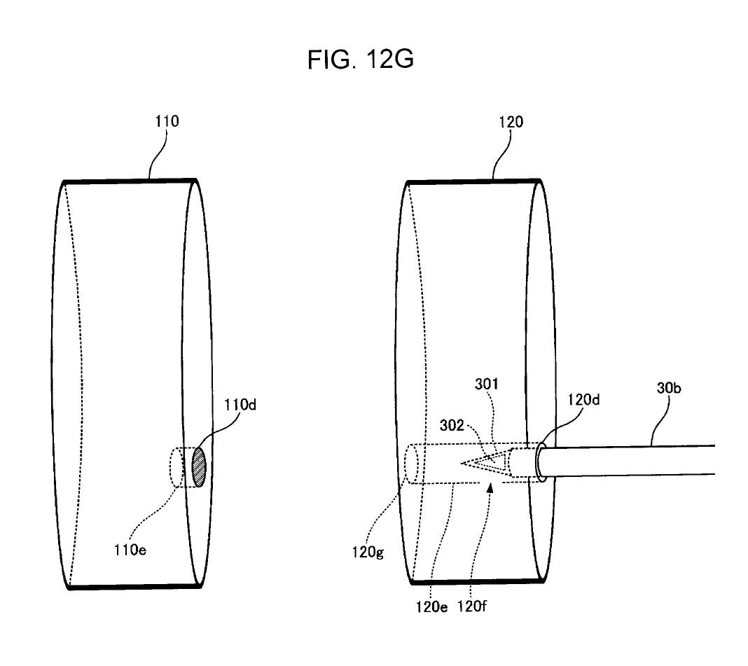

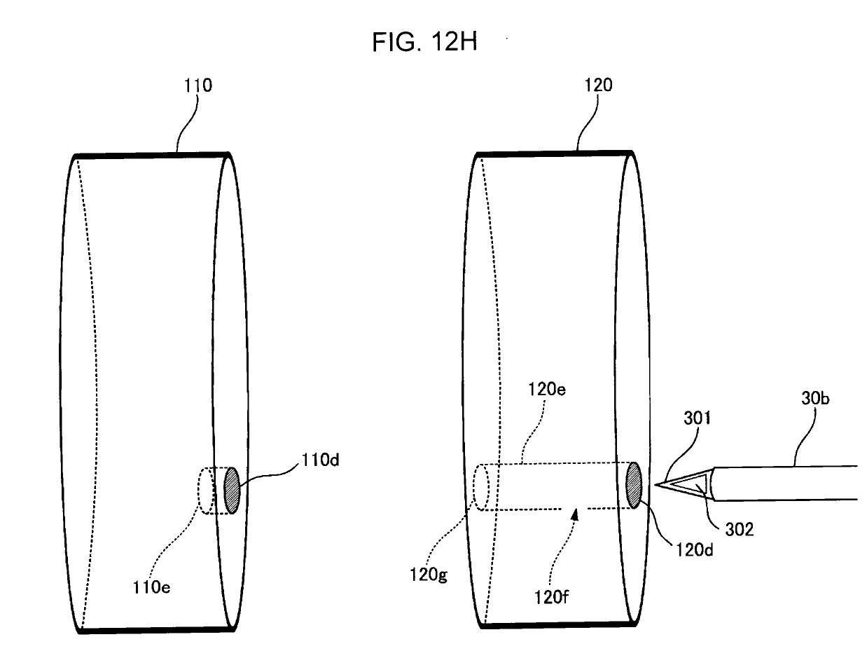

In the present modified example, an example of a configuration for supplying a fluid to the front blocking part 110 and the rear blocking part 120 will be described with reference to FIG. 12A to FIG. 12H.

As shown in FIG. 12A, in addition to the base part 110a and the inflating part 110b which are the same as the above embodiment, the front blocking part 110 comprises a valve 110d. An opening is formed on the face of the insertion opening side (mouth side in the present example) of the subject P in the inflating part 110b, and the valve 110d is fit into this opening. This opening communicates with the internal region of the inflating part 110b. Symbol 110e in FIG. 12C etc. indicates the opening exposed to the inflating part 110b. The valve 110d is configured so as to close the opening. When the opening is in a closed state, the valve 110d restricts the movement of the fluid supplied inside the inflating part 110b to the outside. That is, while in a closed state, the valve 110d functions to prevent the fluid inside the inflating part 110b from leaking.

With regard to the rear blocking part 120, a valve is provided on both faces. That is, an opening is formed on the face of the insertion opening side of the subject P in the inflating part of the rear blocking part 120, and a valve 120d is fit into this opening. In addition, an opening is also formed on the face of the front blocking part 110 side in the inflating part of the rear blocking part 120, and a valve 120g is fit into this opening. These two openings are arranged in opposite-facing positions and communicated with each other via a path 120e formed inside the rear blocking part 120. That is, the two valves 120d and 120g are arranged on both ends of the path 120e that penetrates the rear blocking part 120. The two valves 120d and 120g are configured so as to be capable of closing the openings, respectively. While the openings are in a closed state, the valves 120d and 120g restrict the movement of the fluid that has been supplied inside the inflating part to the outside. That is, while in a closed state, the valves 120d and 120g function to prevent the fluid inside the inflating part of the rear blocking part 120 from leaking. Furthermore, at least a part of the path 120e is exposed inside the inflating part. In the example shown in FIG. 12C etc., an opening 120f that communicates with the path 120e and the inside of the inflating part is formed at part of the path 120e. As another example, a path may be formed from a tubular member in mesh form.

FIG. 12B illustrates the tip part of a tubular member 30b that is inserted inside the second cable part 30B. The tubular member 30b is a hollow member that is flexible as in the second cable part 30B. The base end of the tubular member 30b is connected to the fluid supplying part 48 and used to supply fluid to the front blocking part 110 and the rear blocking part 120. An apex 301 of tapered shape is provided at the tip part of the tubular member 30b. One or more holes 302 are formed on the circumferential face of the apex 301. The hole 302 communicates with the hollow area of the tubular member 30b. As a result, the fluid guided by the tubular member 30b is ejected from the hole 302.

The application mode of the present modified example having a configuration as above will be described with reference to FIGS. 12C to 12H. In the state illustrated in FIG. 12C, the front blocking part 110 and the rear blocking part 120 are in a shrunken state. Furthermore, all the valves 110d, 120d, and 120g are in a closed state. Although not illustrated, the second cable part 30B is provided so as to guide the tubular member 30b to the valves 110d, 120d, and 120g. For example, the second cable part 30B is connected to the opening at which the valve 120d is provided, and is configured so as to connect the opening at which the valve 120d is provided and the opening at which the valve 110d is provided.

The user causes the apex 301 of the tubular member 30b to enter the path 120e via the valve 120d of the rear blocking part 120. Furthermore, the user moves the tubular member 30b forward and guides the apex 301 to the outside of the rear blocking part 120 via the valve 120g. Consequently, the tubular member 30b penetrates the rear blocking part 120 (ref. FIG. 12D). At this time, the valves 120d and 120g alter their shapes or move in response to the passage of the apex 301.

A configuration example of a case in which valves are deformed is described. The valve 120d (120g) is configured from a resilient member, for example, such as rubber, and forms a circular opening (an opening of a shape corresponding to the cross-section of tubular member 30b) that freely alters its size by resilient deformation. As the tubular member 30b moves forward, the tapered apex 301 gradually pushes the opening wider. During this process, due to the action of the resilient deformation, the circumferential face of the tubular member 30b and the edge of the opening are substantially contacted with each other at any time.

A configuration example of the case in which valves are moved is described. The valve 120d (120g) is of a configuration in which a closing member that insulates the inside and outside of the rear blocking part 120 is movably retained by a mechanism such as a hinge etc. As the tubular member 30b moves forward, the closing member is moved by this mechanism. As another configuration example, the valve 120d (120g) may comprise a diaphragm blade mechanism that includes a plurality of vane-shaped members, wherein the superposing regions of the vane-shaped members may be varied. The diaphragm blade mechanism is configured to enlarge the opening size thereof in response to the entry of the tubular member 30b.

From the state shown in FIG. 12D, the user moves the tubular member 30b further forward. Subsequently, the user causes the apex 301 of the tubular member 30b to enter the inside of the front blocking part 110 via the valve 110d of the front blocking part 110. Upon receiving a predetermined operation, the external device 40 starts supplying fluid. The fluid transferred from the fluid supplying part 48 reaches the apex 301 through the hollow area of the tubular member 30b and flows into the inside of the front blocking part 110 (the inflating part 110b) via the hole 302. Consequently, the outer dimensions of the front blocking part 110 (the inflating part 110b) are enlarged. The supply of fluid is conducted until the lumen is substantially occluded by the front blocking part 110 (ref. FIG. 12E).

Subsequently, the user pulls the tubular member 30b out from the front blocking part 110 (ref. FIG. 12F). At this time, the valve 110d returns to its closed state by deformation or movement in response to the retrieval of the apex 301. Furthermore, the user starts pulling out the tubular member 30b. Specifically, the user retrieves the tubular member 30b until reaching a state in which the apex 301 is arranged inside the path 120e of the rear blocking part 120. At this time, the valve 120g returns to its closed state by deformation or movement in response to the passage of the apex 301.

The external device 40 receives a predetermined operation and starts supplying the fluid. The fluid transferred from the fluid supplying part 48 reaches the apex 301 through the hollow area of the tubular member 30b and flows into the inside of the rear blocking part 120 (the inflating part) via the hole 302. Consequently, the outer dimensions of the rear blocking part 120 (the inflating part) are enlarged. The supply of fluid continues until the lumen is substantially occluded by the rear blocking part 120 (ref. FIG. 12G).

Finally, the user pulls the tubular member 30b out from the rear blocking part 120 (ref. FIG. 12H). At this time, the valve 120d deforms or moves to return to a closed state in response to the retrieval of the apex 301.

Following the abovementioned process, as described in the above embodiment, liquid is supplied to the space Eb between the front blocking part 110 and the rear blocking part 120 (ref. Step S5 in FIG. 8), adjustment of the imaging field of vision is carried out (ref. Step S6), and image data is acquired (Step S7).

Liquid may be supplied to the space Eb in Step S5 via the tubular member 30b. In this case, in Step S5, the apex 301 is first arranged inside the space Eb. Subsequently, while this state is maintained, the liquid is supplied to the space Eb via the tubular member 30b from the liquid supplying part 49. It should be noted that if the present example is adopted, the third cable part 30C does not need to be provided.

Once acquisition of the image data is completed, an operation to collect the fluid having been supplied to the front blocking part 110 and the rear blocking part 120 is conducted. This operation is executed via the tubular member 30b in the same way as described above.

As exemplified above, in the ultrasound medical apparatus and ultrasound diagnosis apparatus comprising the same pertaining to the present modified example, an opening is formed on the face of the insertion opening side of the subject P in the inflating part of the front blocking part 110 and/or the rear blocking part 120. Furthermore, in the present modified example, the opening is configured so as to be closable, and is provided with the valves 110d and 120d (restricting member) that restricts movement of fluid from the inside of the inflating part to the outside in the closed state. Furthermore, the liquid supplying part 48 comprises the tubular member 30b (first tubular member) whose tip is inserted inside the inflating part by deforming or moving the valves 110d and 120d in a closed state. The fluid supplying part 48 supplies fluid to the inflating part via the tubular member 30b. In response to the fact that the tubular member 30b has been pulled out from the inflating part with its tip has been inserted, the valves 110d and 120d are deformed or moved so as to return to their closed state. It should be noted that with regard to the rear blocking part 120, an opening is also formed on the face opposite the insertion opening of the subject P, wherein this opening is provided with the valve 120g. the valve 120g acts in the same way as the valves 110d and 120d.

According to the present modified example, a specific configuration is provided for favorably supplying fluid to (and collecting fluid from) the front blocking part 110 and/or the rear blocking part 120.

### Modified Example 5

A modified example is described which is related to processing of supplying liquid to the space Eb between the front blocking part 110 and the rear blocking part 120. In the above embodiment, a case of filling liquid into the space Eb, that is, a case of filling the space Eb with liquid, is specifically described in detail. However, taking the characteristics of an ultrasound examination into consideration, it is not necessary to fill the entire space Eb with liquid as long as the liquid is present in the transmitting and receiving path of ultrasound waves. In other words, it is sufficient that liquid is distributed such that attenuation of ultrasound waves and unnecessary reflections do not occur inside the lumen.

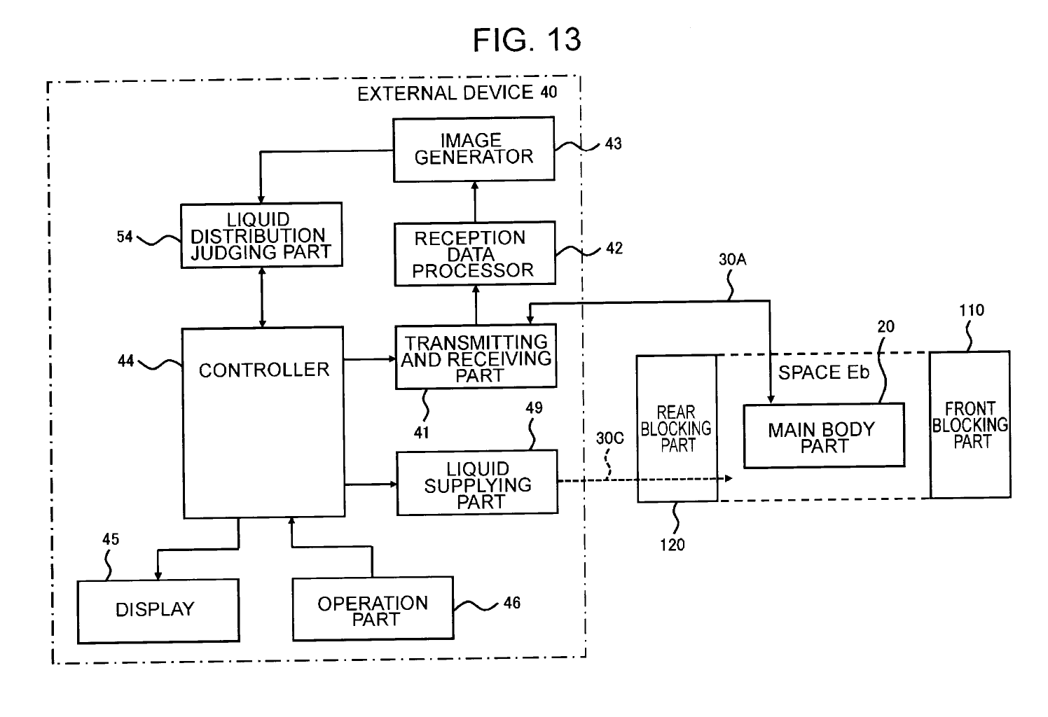

A configuration example pertaining to the present modified example is depicted in FIG. 13. In the present modified example, the external device 40 comprises a liquid distribution judging part 54 in addition to the configuration shown in FIG. 7. In this present modified example, while supplying liquid into the space Eb or after supplying liquid into the space Eb, ultrasound waves are transmitted and received and image data is generated based on data thus acquired. The image data generated by the image generator 43 is sent to the liquid distribution judging part 54.

The liquid distribution judging part 54 judges whether or not the distribution of liquid in the space Eb is appropriate based on the image data generated by the image generator 43. This judgment process includes, for example: a process to obtain information indicating the depiction state (depiction state information) of an observation object (the heart and surrounding vascular system H) based on image data; and a process to judge the appropriateness based on the depiction state information obtained.

The process of obtaining the depiction state information is conducted by analyzing image data. This process is conducted, for example, as described below. First, the liquid distribution judging part 54 extracts partial data of image data corresponding to the observation object. This extracting process may include image processing such as threshold processing, filtering processing, etc. Furthermore, the extracting process may include image processing such as pattern matching based on the shape of the observation object. The liquid distribution judging part 54 calculates an evaluation value indicating the level of image quality based on the partial data obtained by the extracting process. Arbitrary image evaluation technology may be applied in this processing. The evaluation value thus obtained is used as the depiction state information.

Once the depiction state information is obtained, the liquid distribution judging part 54 determines whether or not the distribution of the liquid in the space Eb is appropriate based on the depiction state information. If the evaluation value of the image quality is included in the depiction state information, the liquid distribution judging part 54 compares this evaluation value and a predetermined threshold value, for example. Subsequently, if the evaluation value is equal to or greater than the threshold value, the liquid distribution judging part 54 judges that the distribution state of the liquid is appropriate, while if the evaluation value is less than the threshold value, the distribution state is judged as inappropriate. In the present example, appropriate distribution of the liquid means that imaging of an observation object is possible with appropriate image quality.

The judgment result by the liquid distribution judging part 54 is sent to the controller 44. The controller 44 controls the output of predetermined notification information based on the judgment result. This notification control includes, for example, control of the display 45 to display predetermined visual information (character information, image information, etc.), or control of causing audio outputting part (not illustrated) to output predetermined audio information (alert sound etc.).

The controller 44 outputs notification information only when the obtained determination result is “inappropriate”, for example. Alternatively, the controller 44 may be configured to output notification information regarding the content corresponding to judgment result when judgment result “appropriate” or “inappropriate” is obtained. Moreover, numerical information such as an evaluation value of image quality may be displayed as character information and/or image information.

As exemplified above, the ultrasound medical apparatus and ultrasound diagnosis apparatus including the same pertaining to present modified example comprise: the liquid distribution judging part 54 that judges whether or not the distribution of the liquid in the space Eb is appropriate based on the image data generated by the image generator 43; and the controller 44 that executes notification control based on the judgment result. Here, the liquid distribution judging part 54 corresponds to a “second judging part” and the controller 44 (and the display 45, the audio outputting part, etc.) corresponds to a “second notifying part.”

According to the configuration pertaining to the present modified example, it is possible to judge whether or not liquid is appropriately distributed in the space Eb between the front blocking part 110 and the rear blocking part 120, that is, whether or not the observation object may appropriately be imaged. Thereby, the process of supplying liquid into the space Eb may favorably be supported. Moreover, the operation of adjusting the imaging field of vision may be made easier by arbitrarily combining the processing described above and processing of changing the location and/or the orientation of the ultrasound transducer 21.

### Second Embodiment

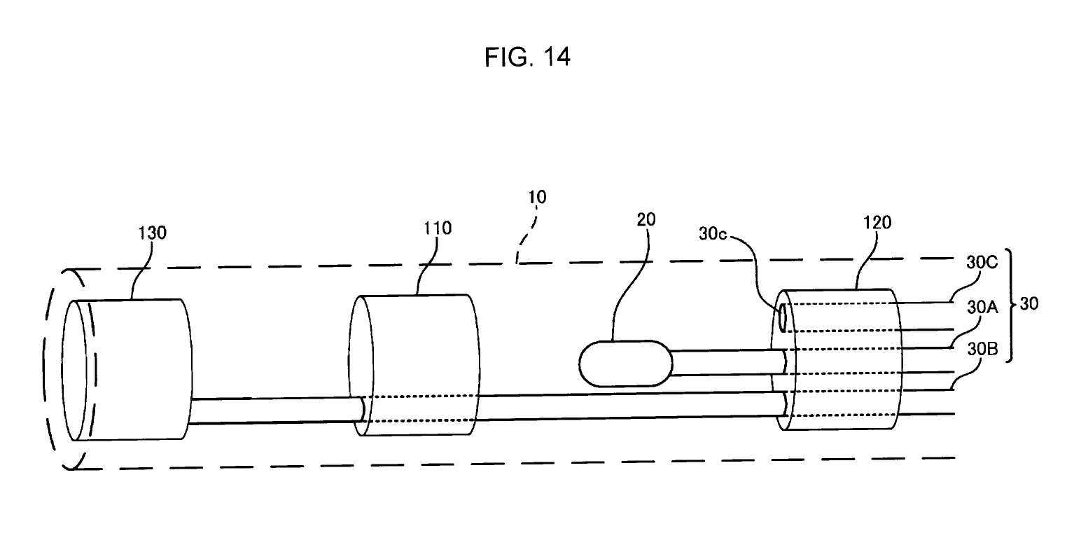

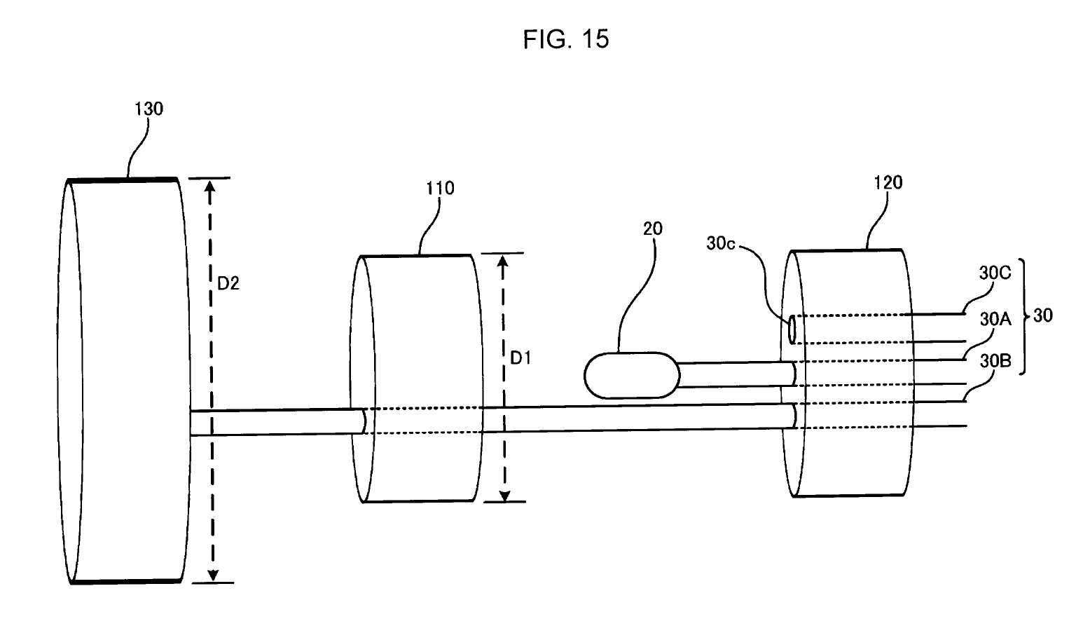

An ultrasound medical apparatus and an ultrasound diagnosis apparatus comprising the same pertaining to the second embodiment will be described with reference to FIGS. 14 to 18J. In the present embodiment, a configuration is described for ensuring the arrangement of the ultrasound medical apparatus at a desired location inside the lumen. Hereinafter, an example of the case in which the ultrasound medical apparatus is arranged in an esophagus is described. It should be noted that if not specifically mentioned, arbitrary configurations among those described in the first embodiment and the modified examples thereof are applicable in the present embodiment.

### (Configuration)

As in the first embodiment, the ultrasound medical apparatus pertaining to the present embodiment comprises the main body part 20, the front blocking part 110, the rear blocking part 120 and the cable part 30 (ref. FIG. 2, etc.). Furthermore, this ultrasound diagnosis apparatus comprises a stabilizer 130 such as that illustrated in FIG. 14. Moreover, in addition to such ultrasound medical apparatus, the ultrasound diagnosis apparatus pertaining to the present embodiment comprises the external device 40 which is the same as that in the first embodiment (ref. FIG. 7, etc.).

The stabilizer 130 is arranged on the side opposite the insertion opening of the subject P with respect to the front blocking part 110. That is, while in an inserted state, the stabilizer 130 is arranged at the location most distant from the insertion opening of the subject P. The stabilizer 130 is configured so as to be able to substantially occlude the lumen by the change in its outer dimensions as in the front blocking part 110 etc.

As in the front blocking part 110, for example, in FIG. 5A, the stabilizer 130 comprises a base part and an inflating part. The base part is formed in a columnar shape, and the inflating part is formed in a cylindrical shape around the base part. The inflating part is configured such that the outer dimensions are enlarged by inflating in the event of receiving a supply of fluid from outside. On the other hand, the inflating part shrinks in the event of receiving discharge of the fluid filled inside the inflating part. The fluid is supplied to the stabilizer 130 by the fluid supplying part 48 of the external device 40. Further, fluid supplied to the stabilizer 130 may be circulated as in the case of the front blocking part 110 etc.

The maximum outer dimensions of the stabilizer 130 may be designed to be larger than the maximum outer dimensions of the front blocking part 110 and/or the rear blocking part 120 (ref. FIG. 15: D2>D1). Furthermore, as in the front blocking part 110 etc., the stabilizer 130 may be of any one of the following configurations in which: (1) An abutting part that abuts the wall face of the lumen comprises a resilient member; (2) An abutting part comprises a member that increases its viscosity as water is added; (3) An abutting part comprises a protrusion part that protrudes toward the wall face of the lumen; and (4) At least the tip of the protrusion part is formed in a needle shape. Furthermore, resistive processing against body fluids such as gastric juices (acid resistance processing etc.) may be applied at least on the face, of the stabilizer 130, on the side opposite the insertion opening of the subject P. That is, resistive processing may be applied on a part where gastric juices etc. might adhere thereto.

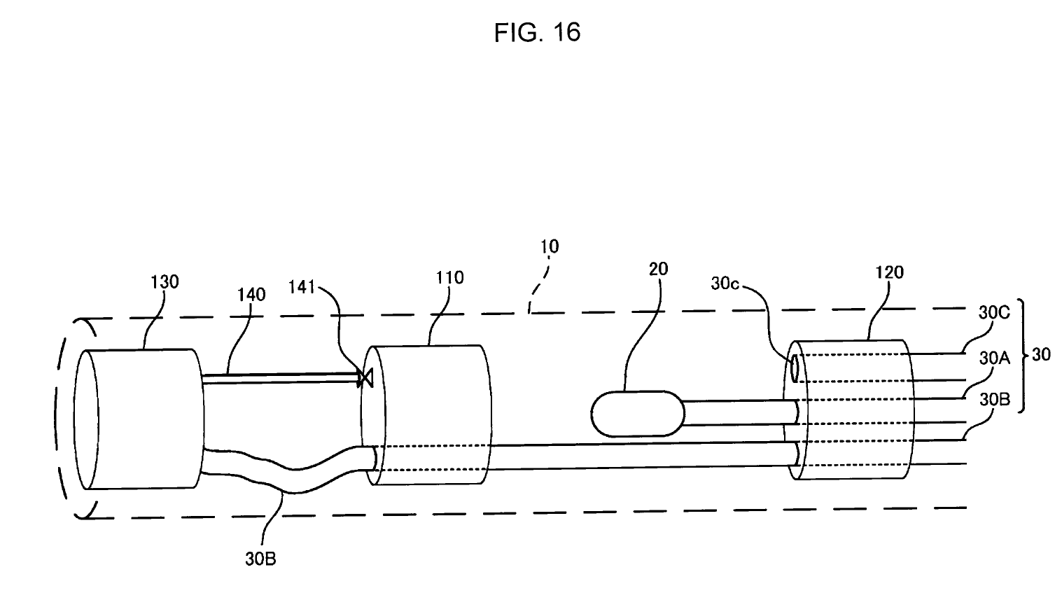

A connecting member connecting the front blocking part 110 and the stabilizer 130 may be provided. This connecting member is of, for example, a predetermined length, with one end thereof connected to the face on the stabilizer 130 side of the front blocking part 110 and the other end connected to the face on the front blocking part 110 side of the stabilizer 130. An example of the connecting member is illustrated in FIG. 16. A connecting member 140 depicted in this figure is connected to an engagement part 141 provided on the front blocking part 110. Furthermore, the connecting member 140 is connected to the stabilizer 130 via a similar engagement part. The front blocking part 110 and the stabilizer 130 connected via the connecting member 140 in this way are inserted into the lumen together with the main body part 20 and the rear blocking part 120. In the present example, a plurality of connecting members 140 of different lengths may be selectively adopted. Consequently, the distance between the front blocking part 110 and the stabilizer 130 may be changed. It should be noted that the connecting member 140 to be adopted is arbitrarily selected according to the body shape etc. of the subject P.

In another configuration example of the connecting member, as in the Modified Example 1 of the first embodiment, it is possible to provide a configuration whereby the interval may be changed while in a state arranged in the lumen.

### (Application Mode)

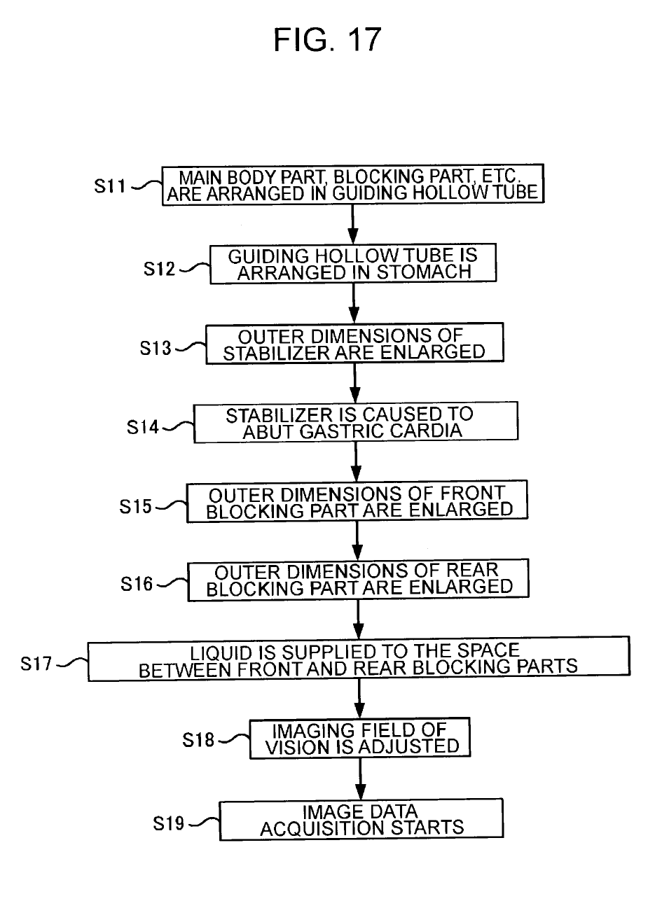

The application mode of the ultrasound diagnosis apparatus pertaining to the embodiment is described. One example of the application modes of the ultrasound diagnosis apparatus 1 is shown in FIG. 17.

(S11: Main Body Part, Blocking Part, Etc. Are Arranged Inside Guiding Hollow Tube)

First, the user arranges the series of the main body part 20, the front blocking part 110, the rear blocking part 120, the stabilizer 130, and the cable part 30 inside the guiding hollow tube 10.

(S12: Guiding Hollow Tube is Inserted into Stomach)

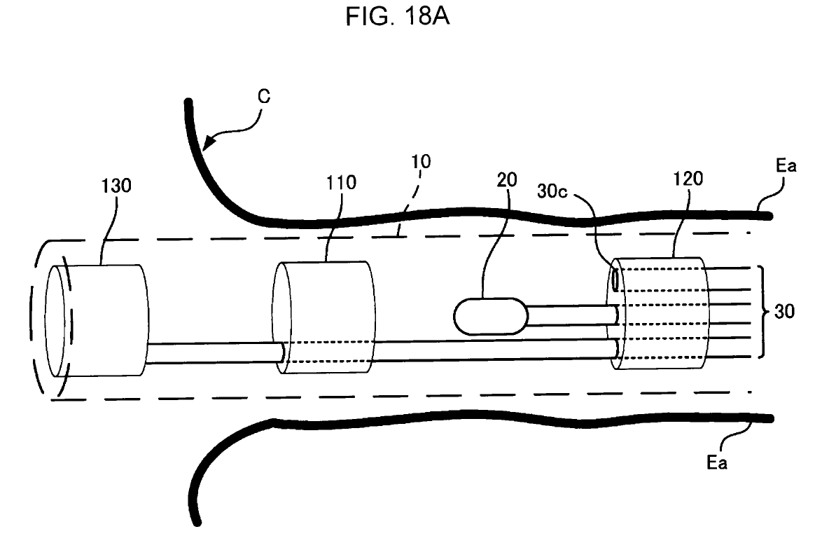

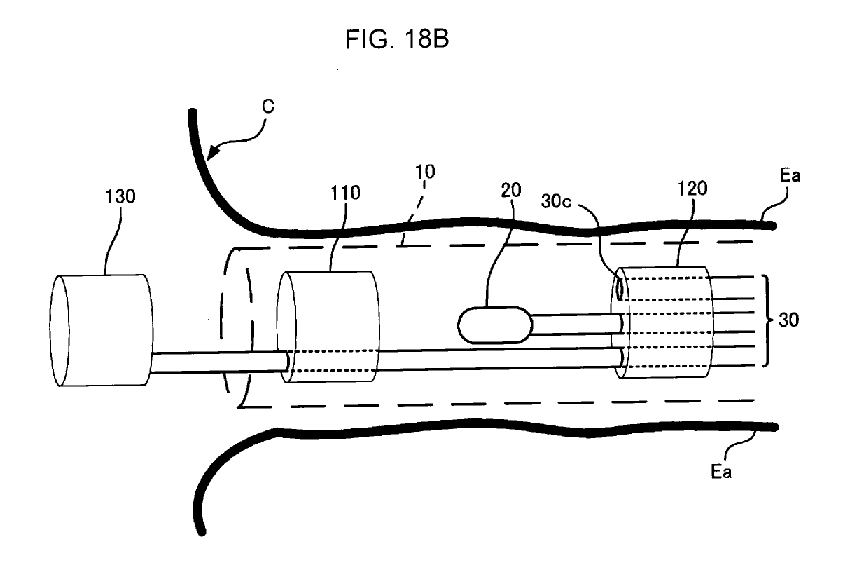

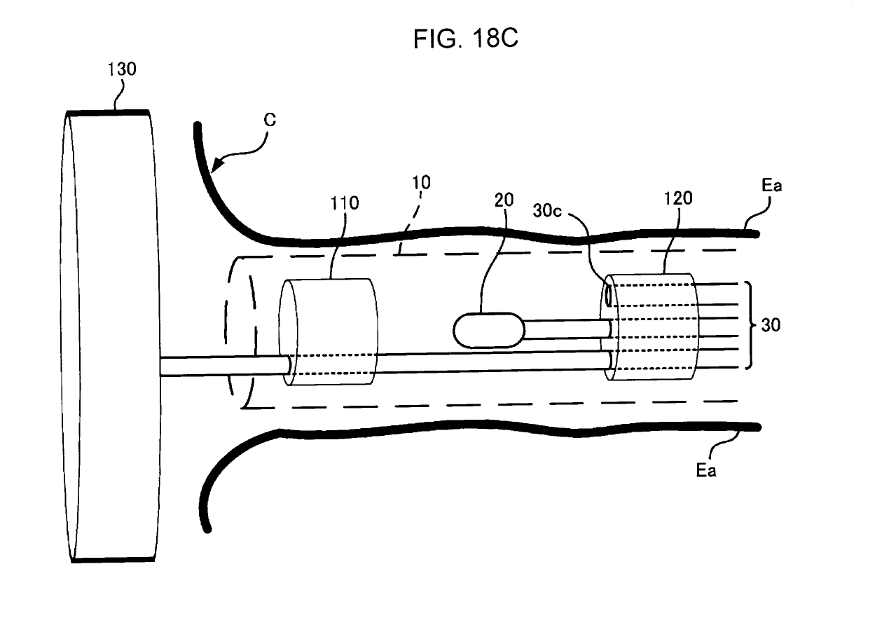

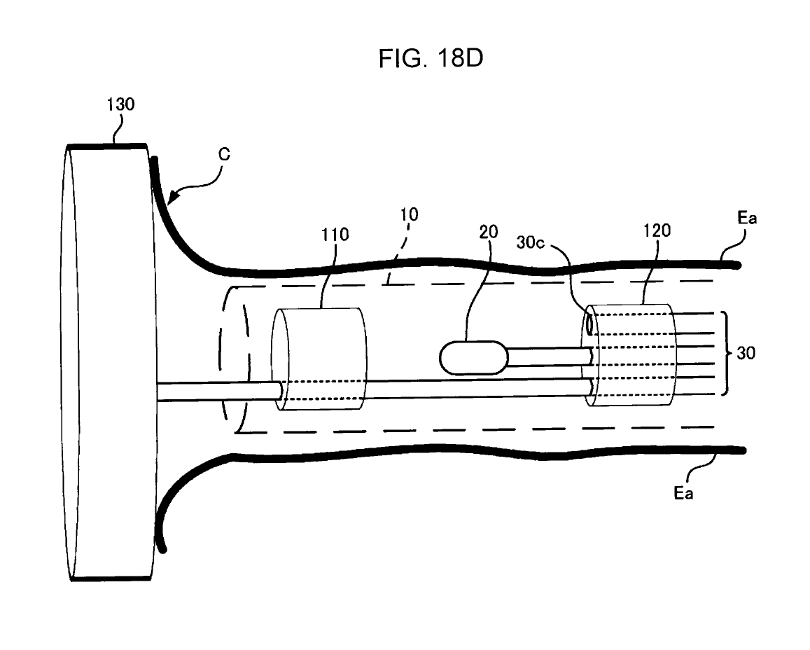

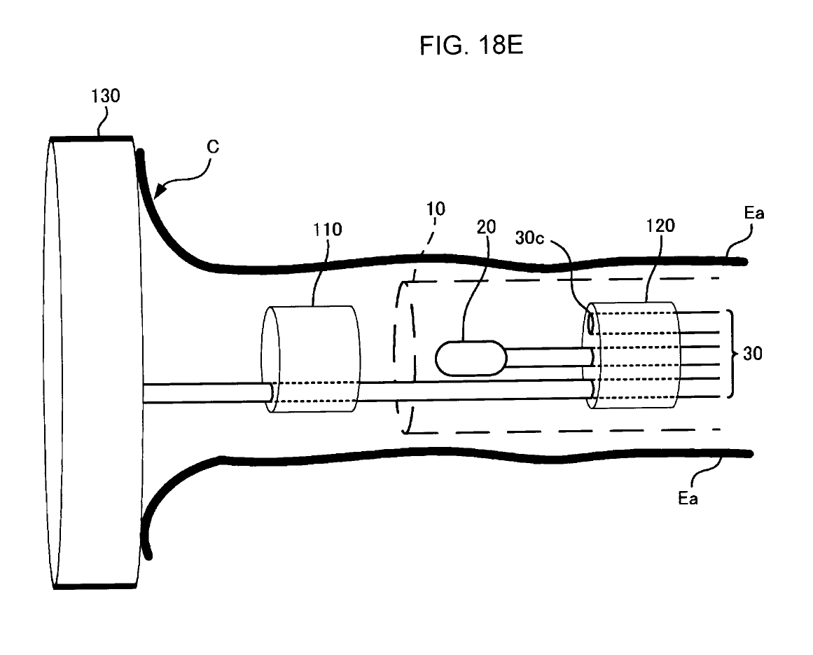

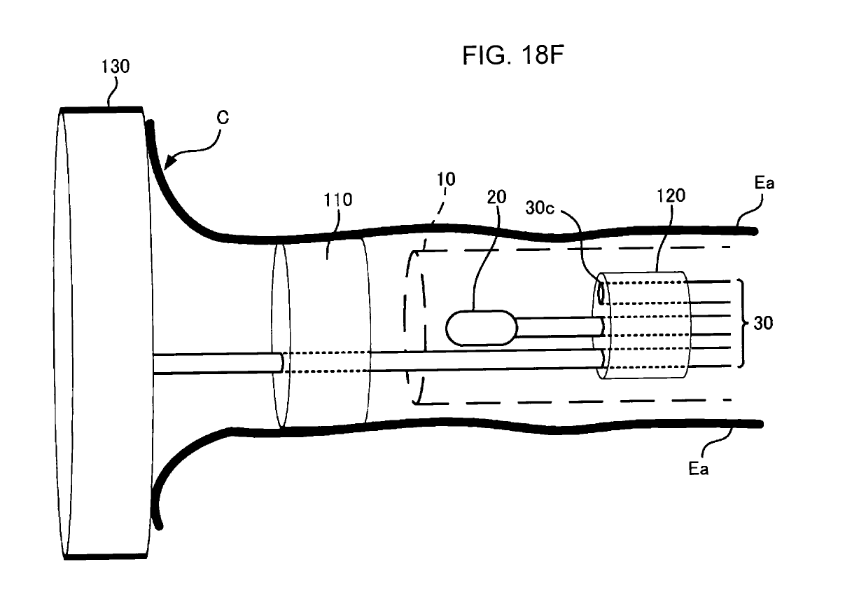

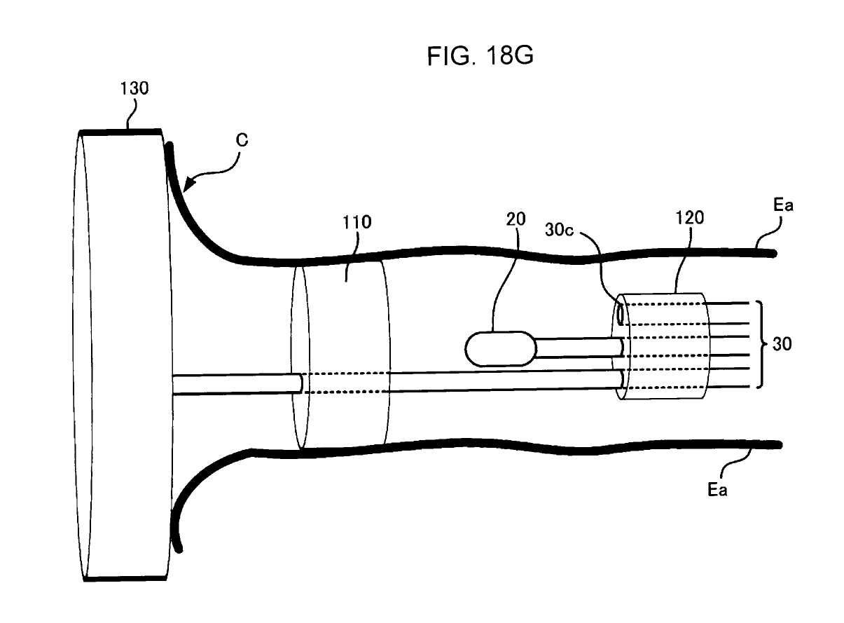

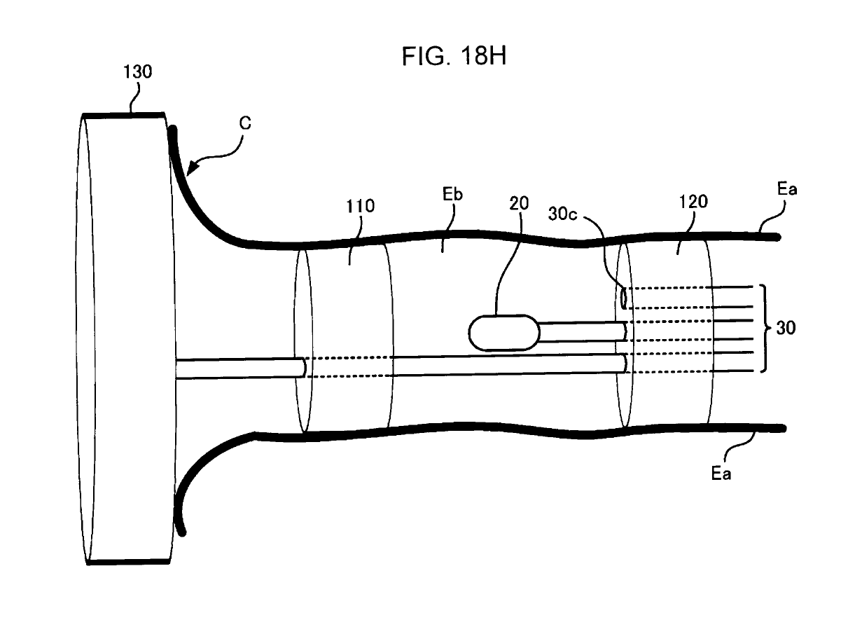

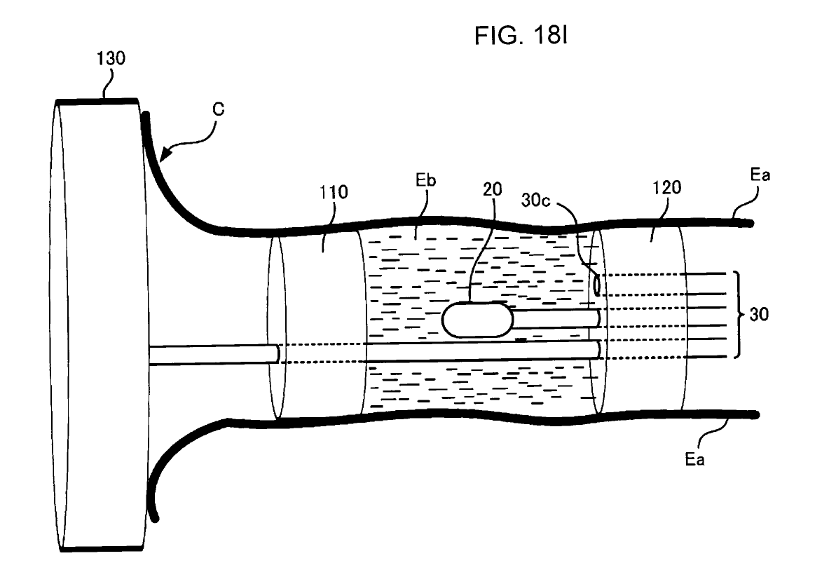

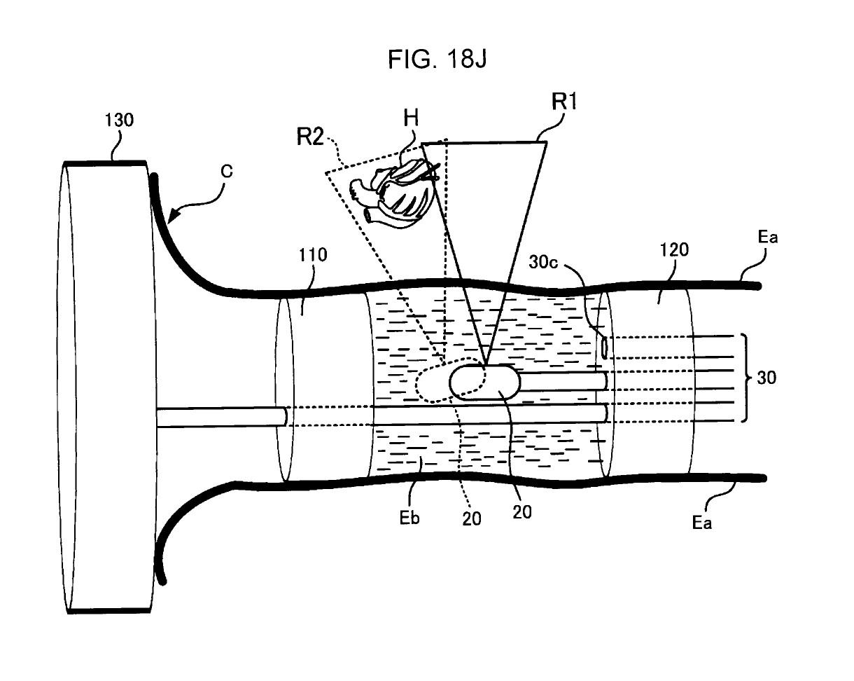

Next, the user inserts the guiding hollow tube 10 in which the main body part 20 etc. have been arranged in Step S11 from the insertion opening of the subject P. In this application mode, the guiding hollow tube 10 is inserted such that at least the stabilizer 130 is arranged inside the stomach. This state is illustrated in FIG. 18A. It should be noted that symbol C in this figure indicates the gastric cardia of the subject P.

### (S13: Outer Dimensions of Stabilizer are Enlarged)

Once the guiding hollow tube 10 is inserted into the stomach in Step S12, the user pulls the guiding hollow tube 10 slightly out. The length to be pulled out is approximately to the extent sufficient to at least expose the stabilizer 130 in the stomach (ref. FIG. 18B). Furthermore, the user performs an operation for enlarging the outer dimensions of the stabilizer 130 via the operation part 46. In response to this operation, the controller 44 controls the fluid supplying part 48 to supply fluid into the inflating part of the stabilizer 130. Consequently, the stabilizer 130 expands inside the stomach. This state is illustrated in FIG. 18C.

### (S14: Stabilizer is Caused to Abut Gastric Cardia)

Once the outer dimensions of the stabilizer 130 are enlarged in Step S13, the user slowly pulls the guiding hollow tube 10 out. As this pulling operation proceeds, resistance comes into play at a certain point. This indicates that the stabilizer 130 has come to abut the gastric cardia C. This state is illustrated in FIG. 18D. It should be noted that the present example is designed such that the size of the stabilizer 130 when inflated is larger than the inner diameter of the esophagus E.

### (S15: Outer Dimensions of Front Blocking Part are Enlarged)

When the stabilizer 130 comes to abut the gastric cardia C in Step S14, the user pulls the guiding hollow tube 10 slightly out. The length to be pulled out is approximately to the extent sufficient to at least expose the front blocking part 110 in the esophagus E (ref. FIG. 18E).

Furthermore, the user performs an operation for enlarging the outer dimensions of the front blocking part 110 via the operation part 46. In response to this operation, the controller 44 controls the fluid supplying part 48 to supply fluid to the inflating part 110b of the front blocking part 110. Consequently, the front blocking part 110 substantially occludes the esophagus E. This state is illustrated in FIG. 18F. In this step, as in the first embodiment, the external device 40 or the user may control the amount of fluid supply.

### (S16: Outer Dimensions of Rear Blocking Part are Enlarged)

Once the outer dimensions of the front blocking part 110 are enlarged in step S15, the user pulls the guiding hollow tube 10 slightly out so as to expose the main body part 20 and the rear blocking part 120 in the esophagus E (ref. FIG. 18G). It should be noted that the entire guiding hollow tube 10 may be pulled out in this stage.

Furthermore, the user performs an operation for enlarging the outer dimensions of the rear blocking part 120 via the operation part 46. In response to this operation, the controller 44 controls the fluid supplying part 48 to supply fluid to the inflating part of the rear blocking part 120. Consequently, the rear blocking part 120 substantially occludes the esophagus E. This state is illustrated in FIG. 18H. It should be noted that control of the amount of fluid supply may be carried out in the same way as Step S15.

### (S17: Liquid is Supplied to Space Between Front and Rear Blocking Parts)

When the rear blocking part 120 substantially occludes the esophagus E in Step S16, the space E between the front blocking part 110 and the rear blocking part 120 becomes a substantially closed space. The user carries out an operation for supplying liquid to the space Eb via the operation part 46. In response to this operation, the controller 44 controls the liquid supplying part 49 to supply liquid to the space Eb. The state in which the space Eb is filled with liquid is illustrated in FIG. 18I.

As in the first embodiment, control of the amount of liquid supply is conducted by the external device 40 or by the user.

### (S18: Imaging Field of Vision is Adjusted)

When liquid is supplied to the space Eb in Step S17, the user or the controller 44 performs adjustment of the imaging field of vision. An outline of the operation to adjust the imaging field of vision is illustrated in FIG. 18J. The user or the controller 44 moves the position of the main body part 20 from the initial position (indicated by the solid line) to a desired position and orientation (indicated by the dotted line). Consequently, the range subject to transmission and reception of ultrasound waves is changed from an initial range R1 to a desired range R2. The desired range R2 includes at least a part of the heart and the surrounding vascular system H that is an observation target.

### (S19: Acquisition of Image Data is Started)

Once the imaging field of vision is adjusted in Step S18, as in the first embodiment, the acquisition of image data of the heart and the surrounding vascular system H is started. When the acquisition of image data is completed, the fluid inside each of the front blocking part 110, the rear blocking part 120 and the stabilizer 130 is collected, thereby shrinking their outer dimensions. Subsequently, these and the main body part 20 are pulled out from inside the body. Thus far, the application mode has been described and ends here.

### (Effects)

The effects of the ultrasound diagnosis apparatus and the ultrasound medical apparatus comprising the same pertaining to the embodiment are described.

The ultrasound medical apparatus (ultrasound probe) included in the ultrasound diagnosis apparatus comprises the main body part 20, the front blocking part 110 (first blocking part), the rear blocking part 120 (second blocking part), the stabilizer 130, and the liquid supplying part 49. The main body part 20 comprises the ultrasound transducer 21 that transmits and receives ultrasound waves, and is inserted into the lumen (esophagus E) of the subject P. the front blocking part 110 is inserted into the lumen (esophagus E) and arranged on the side opposite (stomach side) the insertion opening (mouth) of the subject P with respect to the main body part 20. Furthermore, the front blocking part 110 is configured so as to substantially occlude the lumen (esophagus E) by means of changing the outer dimensions thereof. The rear blocking part 120 is inserted into the lumen (esophagus E) and arranged on the insertion opening (mouth) side with respect to the main body part 20. Furthermore, the rear blocking part 120 is configured so as to substantially occlude the lumen (esophagus E) by means of changing the outer dimensions thereof. The stabilizer 130 is inserted into the lumen (stomach) and arranged on the side opposite the main body part 20 with respect to the front blocking part 110. Furthermore, the stabilizer 130 is configured so as to be capable of fixing the position in the lumen by means of changing the outer dimensions thereof. The liquid supplying part 49 is configured so as to supply liquid into the space Eb between the front blocking part 110 and the rear blocking part 120.

According to such ultrasound medical apparatus and ultrasound diagnosis apparatus comprising the same, as in the first embodiment, high quality images of a desired site inside the body may be acquired with low invasiveness. Furthermore, the position of the main body part 20 etc. may be substantially fixed by the action of the stabilizer 130, thereby ensuring the arrangement of the main body part 20 at a desired location. Consequently, the imaging field of vision may be stably maintained.

In the present embodiment, it is possible to design such that the maximum outer dimensions of the stabilizer 130 are larger than the respective maximum outer dimensions of the front blocking part 110 and the rear blocking part 120. This configuration is effective in the case in which the sizes of the lumen differ greatly depending on the sites such as an esophagus and a stomach in the above application mode, for example.

In the present embodiment, a mechanism may be provided for changing the interval between the front blocking part 110 and the stabilizer 130. This mechanism may comprise, for example, the connecting member and engagement part 141 illustrated in FIG. 16. Alternatively, as in the Modified Example 1 in the first embodiment, this mechanism may comprise a mechanism whereby the interval may be changed while in a state arranged in the lumen.

In the present embodiment, resistive processing against body fluid may be applied at least on the face, of the stabilizer 130, on the side opposite the main body part 20. As a result of this configuration, denaturation (deformation or damage) of the stabilizer 130 due to body fluid (gastric juices etc.) may be prevented.

In the present embodiment, the stabilizer 130 may be configured so as to substantially occlude the lumen. The configuration for this may be similar to that of the front blocking part 110 etc., for example. With such a configuration, it becomes possible to prevent the adhesion of body fluid onto the front blocking part 110 etc. (for example, the reverse flow of gastric juices may be prevented). It should be noted that if the stabilizer 130 to be adopted does not have a occluding action, resistive processing against body fluid may be applied at least on the face of the stabilizer 130 side of front blocking part 110.

In the present embodiment, the stabilizer 130 may comprise an inflating part whose outer dimensions are enlarged by means of inflation upon receipt of a fluid supply from the fluid supplying part 48. According to this configuration, the enlargement of the outer dimensions of the stabilizer 130 may be realized by a simple structure. It should be noted that the configuration to enlarge the outer dimensions of the stabilizer 130 is not limited to this. For example, as described in the first embodiment, a foldable-type arm capable of bending and extending or a diaphragm blade mechanism comprising a plurality of blade-like members capable of changing the superposing regions may be adopted.

In addition to the abovementioned ultrasound medical apparatus, the ultrasound diagnosis apparatus pertaining to the present embodiment has a function to generate image data by processing echo signals based on reflected waves received by the ultrasound transducer 21. This image generating function is realized by an “image generating part” comprising the reception data processor 42 and the image generator 43.

### Third Embodiment

The Modified Example 4 of the first embodiment describes a configuration for supplying fluid to the front blocking part 110 and the rear blocking part 120 using the tubular member that can be inserted and removed with respect to them. The third embodiment describes a case in which liquid is supplied to the space (the space Eb illustrated in FIG. 4 etc.) between the front blocking part 110 and the rear blocking part 120 using a similar configuration to the Modified Example 4. It should be noted that unless specifically stated, it is possible to apply, to the present embodiment, an arbitrary configuration among those described in the first embodiment and its modified examples as well as the second embodiment. Hereinafter, an example of an apparatus pertaining to the present embodiment will be described with reference to the figures pertaining to the first embodiment.

### (Configuration)

The ultrasound medical apparatus and the ultrasound diagnosis apparatus comprising the same pertaining to the present embodiment have similar configuration to the first embodiment and, in particular, have the configuration illustrated in FIG. 12A to FIG. 12H. Specifically, as in the configuration illustrated in FIG. 7 etc., the ultrasound diagnosis apparatus pertaining to the present embodiment comprises at least the main body part 20, the front blocking part 110, the rear blocking part 120, the fluid supplying part 48 and the liquid supplying part 49. It should be noted that the cable part 30 connecting the main body part 20 and the external device 40 is also provided.

Furthermore, in addition to the components of the concerned ultrasound medical apparatus, the ultrasound diagnosis apparatus pertaining to the present embodiment comprises the image generator 43, and may further comprise the liquid distribution judging part 54 (second judging part) as well as the controller 44 (along with the display 45, audio outputting part, etc.) (second notifying part).

As in the first embodiment, the main body part 20 comprises the ultrasound transducer 21 that transmits and receives ultrasound waves, and is inserted into the lumen of the subject P (ref. FIG. 7 etc.)

As in the first embodiment, the front blocking part 110 in the present embodiment is inserted into the lumen of the subject P, arranged on the side opposite the insertion opening (mouth, anus, etc.) of the subject P with respect to the main body part 20, and configured such that the lumen may be substantially occluded by changing the outer dimensions thereof. Furthermore, in the front blocking part 110 in the present embodiment, a shieldable opening (first opening) is provided on the face of the insertion opening side thereof. The face on the insertion opening side means the face on the main body part 20 side. Furthermore, the first opening may be an opening that is configured so as to be closable by means of the valve 110d illustrated in FIG. 12A etc.

As in the first embodiment, the rear blocking part 120 in the present embodiment is inserted into the lumen of the subject P, arranged on the insertion opening side with respect to the main body part 20, and configured so as to substantially occlude the lumen by changing its outer dimensions. Furthermore, a shieldable opening (second opening) is provided on the face, of rear blocking part 120 in the present embodiment, of the insertion opening side while a shieldable opening (third opening) is also provided on its opposite face. In addition, this rear blocking part 120 is provided with a path connecting the second and third openings (first path). As a specific example of such a configuration, the rear blocking part 120 illustrated in FIG. 12C etc. is cited. That is, the second opening may be an opening configured so as to be closable by means of the valve 120d, the third opening may be an opening configured so as to be closable by means of the valve 120g, and the first path may be the path 120e penetrating the rear blocking part 120.

The fluid supplying part 48 of the present embodiment is described. The fluid supplying part 48 (the tubular member 30b extending therefrom) is capable of being inserted and removed with respect to each of the front blocking part 110 and the rear blocking part 120, and is configured so as to supply fluid while in a state in which the tubular member 30b is inserted. This configuration is described in more detail. First, the fluid supply to the front blocking part 110 is described. The tip (the apex 301) of the tubular member 30b extending from the fluid supplying part 48 is configured so as to be capable of being inserted/retrieved with respect to the first opening (the valve 110d) of the front blocking part 110. The fluid supplying part 48 supplies fluid to the front blocking part 110 while in a state in which the apex 301 of the tubular member 30b is arranged inside the front blocking part 100 through the valve 110d. Consequently, the outer dimensions of the front blocking part 110 are enlarged. Next, the supply of fluid to the rear blocking part 120 is described. The tubular member 30b is configured so as to be capable of being inserted/retrieved with respect to the path 120e of the rear blocking part 120. When the apex 301 of the tubular member 30b is in a state arranged inside the path 120e, that is, when the apex 301 is in a state arranged inside the rear blocking part 120 through the valve 120d, the fluid supplying part 48 supplies fluid to the rear blocking part 120. Consequently, the outer dimensions of rear blocking part 120 are enlarged.

As in the first embodiment, the liquid supplying part 49 of the present embodiment has a function to supply liquid into the space Eb between the front blocking part 110 and the rear blocking part 120.

It should be noted that in this embodiment, a tubular member for supplying fluid to the front blocking part 110 and the rear blocking part 120 and a tubular member for supplying liquid into the space Eb may be the same or different. A configuration using two different tubular members is, for example, illustrated in FIG. 2. That is, in the configuration illustrated in FIG. 2, the second cable part 30B and the third cable part 30C are separately provided, wherein the second cable part 30B is a tubular member for supplying fluid to the front blocking part 110 and the rear blocking part 120 and the third cable part 30C is a tubular member for supplying liquid into the space Eb. As one example of the configuration pertaining to the present embodiment, it is possible to configure the second cable part 30B illustrated in FIG. 2 to play the same role as the tubular member 30b illustrated in FIG. 12B to FIG. 12H.

As an example of a case in which a common tubular member is used for fluid supply and liquid supply, there is a configuration of executing a switching operation of the fluid supplying part 48 and the liquid supplying part 49. This switching operation is executed by the controller 44. In this case, it is possible to provide a switching valve for exclusively switching the flow into tubular member 30 at the position where a path extending from the fluid supplying part 48 and a path extending from the liquid supplying part 49 merge. The operation of this switching valve is controlled by the controller 44.

As another example of a case in which the fluid supply and liquid supply are conducted using a common tubular member, a double pipe may be used. For example, the tubular member 30b may be configured such that the tip of a tubular member extending from the liquid supplying part 49 is arranged inside the tip of a tubular member extending from the fluid supplying part 48. Conversely, the tubular member 30b may be configured such that the tip of a tubular member extending from the fluid supplying part 48 is arranged inside the tip of a tubular member extending from the liquid supplying part 49.

### (Application Mode)

An example of the application mode of the ultrasound diagnosis apparatus pertaining to the present embodiment will be described with reference to FIG. 12C to FIG. 12H. It should be noted that this application mode is an example of the case in which a common tubular member (tubular member 30b) is used to supply fluid to the front blocking part 110 as well as the rear blocking part 120 and supply liquid to the space Eb. Furthermore, arbitrary items described in the Modified Example 4 of the first embodiment may be applied in this application mode.

In the state shown in FIG. 12C, the front blocking part 110 and the rear blocking part 120 are in a shrunken state. Furthermore, all valves (openings) 110d, 120d, and 120g are in a closed state. Although not illustrated, the second cable part 30B is provided so as to guide the tubular member 30b to the valves 110d, 120d, and 120g.

The user inserts the apex 301 of the tubular member 30b into the path 120e through the valve 120d of the rear blocking part 120. Furthermore, the user moves the tubular member 30b forward and guides the apex 301 outside of the rear blocking part 120 through the valve 120g. Consequently, the tubular member 30b penetrates the rear blocking part 120 (ref. FIG. 120D). At this time, the valves 120d and 120g are deformed or moved in response to the passage of the apex 301. The deformation or movement aspects of the valves 120d and 120g may be the same as the Modified Example 4 of the first embodiment.

From the state shown in FIG. 12D, the user moves the tubular member 30b further forward. Subsequently, the user causes the apex 301 of the tubular member 30b to enter the front blocking part 110 through the valve 110d of the front blocking part 110. Upon receiving a predetermined operation, the external device 40 starts supplying fluid. The fluid transferred from the fluid supplying part 48 reaches the apex 301 through the hollow area of the tubular member 30b which is the tip thereof and flows into the front blocking part 110 (the inflating part 110b) through the hole 302. Consequently, the outer dimensions of the front blocking part 110 (the inflating part 110b) are enlarged. The fluid is supplied until the lumen is substantially occluded by the front blocking part 110 (ref. FIG. 12E).

Subsequently, the user pulls the tubular member 30b out from the front blocking part 110 (ref. 12F). At this time, in response to the retrieval of the apex 301, the valve 110d returns to its closed state by deforming or moving. Furthermore, the user is pulling the tubular member 30b out. Specifically, the user retrieves the tubular member 30b until reaching a state in which the apex 301 is arranged in the path 120e of the rear blocking part 120. At this time, the valve 120g returns to its closed state by deforming or moving in response to the passage of the apex 301.

The external device 40 receives a predetermined operation and starts supplying fluid. The fluid transferred from the fluid supplying part 48 reaches the apex 301 through the hollow area of the tubular member 30b and flows into the rear blocking part 120 (inflating part) through the hole 302. Consequently, the outer dimensions of the rear blocking part 120 (inflating part) are enlarged. The fluid supply continues until the lumen is substantially occluded by the rear blocking part 120 (ref. FIG. 12G). The steps up to this point are the same as those in the Modified Example 4 in the first embodiment. According to the steps described thus far, the space Eb between the front blocking part 110 and the rear blocking part 120 becomes substantially closed.

Next, the user resumes moving the tubular member 30b forward to penetrate the rear blocking part 120. Consequently, the apex 301 of the tubular member 30b is arranged inside the space Eb as shown in FIG. 12D or FIG. 12F. Upon receiving a predetermined operation, the external device 40 starts supplying liquid. The liquid transferred from the liquid supplying part 49 reaches the apex 301 through the hollow area of the tubular member 30b and flows into the space Eb through the hole 302.

Once the supplying of liquid to the space Eb is completed, the user starts pulling the tubular member 30b out to retrieve it from the rear blocking part 120 (ref. FIG. 12H).

After the processes described thus far are conducted, as in the first embodiment, the imaging field of vision is adjusted (ref. Step S6 in FIG. 8), and image data is acquired (Step S7). When the acquisition of image data is completed, the fluid having been supplied to the front blocking part 110 and the rear blocking part 120 is collected. This operation is executed through the tubular member 30b as above.

### (Effect)

The effects of the ultrasound medical apparatus and ultrasound diagnosis apparatus pertaining to the present embodiment are described.

The example of the ultrasound medical apparatus pertaining to the present embodiment comprises the main body part (20), the first blocking part (the front blocking part 110), the second blocking part (the rear blocking part 120), the fluid supplying part (the fluid supplying part 48 and the tubular member 30b) and the liquid supplying part (the liquid supplying part 49 and the tubular member 30b).

The main body part comprises the ultrasound transducer (21) that transmits and receives ultrasound waves, and is inserted into the lumen of the subject (P).

The first blocking part is inserted into the lumen of the subject, arranged on the side opposite the insertion opening (mouth) of the subject with respect to the main body part, and configured to be capable of substantial occluding the lumen by changing its outer dimensions. Furthermore, a closable first opening (an opening with the valve 110d) is provided on the face of the insertion opening side of the subject in the first blocking part.

The second blocking part is inserted into the lumen of the subject, arranged on the insertion opening side with respect to the main body part, and configured to being capable of substantial occluding the lumen by changing its outer dimensions. Furthermore, a closable second opening (an opening with the valve 120d) is provided on the face on the insertion opening side in the second blocking part, while a closable third opening (an opening with the valve 120g) is provided on its opposite face (the face opposite the insertion opening). Further, the second blocking part is provided with a first path (the path 120e) that connects the second and third openings.

The tip part of the fluid supplying part (the tubular member 30b) may be inserted and removed with respect to the first opening. The fluid supplying part supplies fluid in a state in which the tip part is arranged inside the first blocking part through the first opening, enlarging the outer dimensions of the first blocking part. Likewise, the tip part of the fluid supplying part is made to be inserted and removed with respect to the first path. The fluid supplying part supplies fluid in a state in which the tip part is arranged in the first path, enlarging the outer dimensions of the second blocking part.

The liquid supplying part supplies liquid into the space (the space Eb) between the first blocking part and the second blocking part.

According to such an ultrasound medical apparatus, as in the first embodiment, high quality images of a desired region inside the body may be acquired with low invasiveness. Furthermore, according to the present embodiment, a specific configuration is provided for favorably supplying fluid to (and collecting fluid from) the first blocking part and the second blocking part.

The second blocking part in this embodiment may be provided with a penetrating path for supplying liquid to the space Eb. Specifically, it is possible to provide a shieldable opening on the face, of the second blocking part (the rear blocking part 120), of the insertion opening side, a shieldable opening on its opposite face (the face on the opposite side from the insertion opening), and further a second path linking these openings. In this case, the tip part of the liquid supplying part (the tubular member 30b or other tubular members) may be inserted and removed with respect to the second path. Subsequently, the liquid supplying part supplies liquid in a state in which the tip part is arranged in the space Eb through the second path.

Here, the second path may be common with the first path or may be provided separately from the first path. In the above application mode, a case in which the first path (the path 120e) works as the second path is described. On the other hand, if the second path is individually provided, for example, as shown in FIG. 14, it is configured such that the cable part 30C (tubular member) is capable of penetrating the rear blocking part 120. In this case, the cable part 30c has, for example, the same configuration as the path 120e and is guided via the second path provided separately from path 120e to penetrate the rear blocking part 120, and liquid supply to the space Eb is carried out. Each end of this second path may be an opening with a valve.

According to such an embodiment, a specific configuration is provided for favorably supplying liquid to (and collecting liquid from) the space between the first blocking part and the second blocking part.

In the present embodiment, the tip part of the fluid supplying part and the tip part of the liquid supplying part may be configured as a common tubular member. The tubular member 30b in the above application mode is an example of such a common tubular member. Furthermore, as another example of a common tubular member, a double-pipe structure may be used. That is, for the common tubular member, it is possible to adopt a double-pipe structure in which the tubular tip part of one of the fluid supplying part and the liquid supplying part is arranged inside the tubular tip part of the other.

According to such an embodiment, the structure for supplying fluid and liquid may be simplified.

The ultrasound diagnosis apparatus pertaining to the present embodiment comprises an ultrasound medical apparatus pertaining to the present embodiment and the image generating part that processes signals based on reflected waves received by the ultrasound transducer to generate image data. This image generating part comprises, for example, the reception data processor 42 and the image generator 43 (ref. FIG. 7).

According to such an ultrasound diagnosis apparatus, as in the first embodiment, high quality images of a desired region inside the body may be acquired with less invasiveness.

The ultrasound diagnosis apparatus of the present embodiment may comprise arbitrary functions in the first and second embodiments. For example, it is possible to provide: a function to judge whether or not the distribution of liquid in the space between the first blocking part and the second blocking part is appropriate based on the image data generated by the image generating part; and a function to execute notification based on the judgment result. This judging function is realized, for example, by the liquid distribution judging part 54 (second judging part) shown in FIG. 13 and this notification function is realized, for example, by the controller 44 (and the display 45, audio outputting part, etc.) (second notifying part).

### <Supplementary Note>

Some of features of the abovementioned embodiments are shown below.

As a first example, an embodiment may include the following features: an opening is formed on a surface, which is on the insertion opening side, of the expanding part of one or both of the first blocking part and the second blocking part; a restricting member is provided, wherein the restricting member is configured so as to be able to shield the opening, and restricts movement of the fluid from the inside to the outside of the expanding part in a shielding state; the fluid supplying part includes a first tubular member whose tip part is inserted into the expanding part by changing the shape of or displacing the restricting member in the shielding state, and supplies the fluid to the expanding part through the first tubular member; the restricting member changes its shape or moves and returns to the shielding state in response to an event that the first tubular member with its tip being inserted is removed from the expanding part.

As a second example, in an embodiment, the abutting parts of the first and second blocking parts that contact a wall surface of the lumen may include elastic member.

As a third example, in an embodiment, the abutting parts of the first and second blocking parts that contact a wall surface of the lumen may include a member whose viscosity increases by adding water.

As a fourth example, in an embodiment, the abutting parts of the first and second blocking parts that contact a wall surface of the lumen may include a protrusion part that protrudes toward the wall surface.

As a fifth example, in an embodiment, at least a tip of the protrusion part may be configured in an acicular shape.

As a sixth example, in an embodiment, the stabilizer may be provided, wherein the stabilizer is inserted into the lumen, is arranged at the opposite side to the insertion opening across the first blocking part, and is capable of stabilizing a location thereof in the lumen by changing the dimension thereof. Further, the maximum dimension of the stabilizer may be larger than the maximum dimensions of the first and second blocking parts.

As a seventh example, in an embodiment, the mechanism configured for changing the interval between the first blocking part and the stabilizer.

As an eighth example, in an embodiment, body-fluid-resistant treatment may be applied to at least the opposite surface of the stabilizer to the insertion opening.

As a ninth example, in an embodiment, the stabilizer may include the expanding part that expands by receiving the supply of the fluid from the fluid supplying part and enlarges the dimension.

As a tenth example, in an embodiment, the mechanism configured for moving the movable part may be provided. The movable part includes at least the ultrasound transducer of the main body. Further, the movable part may be configured to move the movable part in a direction approaching the first blocking part and in a direction approaching the second blocking part.

As an eleventh example, in an embodiment, the mechanism configured for moving the movable part may be provided. The movable part includes at least the ultrasound transducer of the main body. Further, the movable part may be configured to move the movable part so as to change a direction of transmitting and receiving ultrasonic waves by the ultrasound transducer.

As a twelfth example, in an embodiment, at least part of the cable part may have a structure that changes flexibility thereof when wrenched.

As a thirteenth example, in an embodiment, the second judging part may be configured to obtain depiction state information of an observation target of the subject based on the image data generated by the image generating part processing signals based on echoes received by the ultrasound transducer, and performs the judgment based on the depiction state information.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An ultrasound medical apparatus comprising:
a main body part that includes an ultrasound transducer configured to transmit and receive ultrasonic waves, and is inserted into a lumen of a subject;
a first blocking part that is inserted into the lumen, is arranged at the opposite side to an insertion opening of the subject across the main body part, is capable of substantially occluding the lumen by changing the dimension thereof, and is provided, on a surface of the insertion opening side, with a first opening that can be shielded;
a second blocking part that is inserted into the lumen, is arranged at the insertion opening side across the main body part, is capable of substantially occluding the lumen by changing the dimension thereof, and is provided with a first path connecting a second opening that can be shielded and is provided on a surface of the insertion opening side and a third opening that can be shielded and is provided on the opposite surface thereto;
a fluid supplying part, whose tip part is removably inserted into each of the first opening and the first path, that supplies fluid to enlarge the dimension of the first blocking part in a state in which the tip part is arranged inside the first blocking part through the first opening, and supplies fluid to enlarge the dimension of the second blocking part in a state in which the tip part is arranged inside the first path; and
a liquid supplying part that supplies liquid into a space between the first blocking part and the second blocking part.

2. The ultrasound medical apparatus of claim 1, wherein
the second blocking part is provided with a second path connecting a shieldable opening that is provided on the surface of the insertion opening side and a shieldable opening that is provided on the opposite side thereto, and
a tip part of the liquid supplying part can be inserted into the second path and the liquid supplying part supplies the liquid in a state in which this tip part is arranged in the space through the second path.

3. The ultrasound medical apparatus of claim 2, wherein the first path serves also as the second path.

4. The ultrasound medical apparatus of claim 3, wherein the tip part of the fluid supplying part and the tip part of the liquid supplying part are configured as a common tubular member.

5. The ultrasound medical apparatus of claim 4, wherein the common tubular member is configured as a double pipe in which a tubular tip part of one of the fluid supplying part and the liquid supplying part is arranged inside a tubular tip part of the other.

6. An ultrasound medical apparatus comprising:
a main body part that includes an ultrasound transducer configured to transmit and receive ultrasonic waves, and is inserted into a lumen of a subject;
a first blocking part that is inserted into the lumen, is arranged at the opposite side to an insertion opening of the subject across the main body part, and is capable of substantially occluding the lumen by changing the dimension thereof;
a second blocking part that is inserted into the lumen, is arranged at the insertion opening side across the main body part, and is capable of substantially occluding the lumen by changing the dimension thereof;
a mechanism configured for varying an interval between the first blocking part and the second blocking part; and
a liquid supplying part that supplies liquid into a space between the first blocking part and the second blocking part.

7. The ultrasound medical apparatus of claim 6, further comprising a fluid supplying part that supplies fluid, wherein
at least one of the first blocking part and the second blocking part includes an expanding part that expands by receiving the supply of the fluid from the fluid supplying part and enlarges the dimension.

8. The ultrasound medical apparatus of claim 1, wherein
the liquid supplying part includes a tubular member that passes through the second blocking part, wherein an opening at a tip of the tubular member is exposed to the space between the first blocking part and the second blocking part, and
the liquid supplying part supplies the liquid to the space through the tubular member.

9. The ultrasound medical apparatus of claim 1, wherein the liquid supplying part supplies new liquid while collecting liquid that has already supplied to the space.

10. The ultrasound medical apparatus of claim 1, further comprising a stabilizer that is inserted into the lumen, is arranged at the opposite side to the insertion opening across the first blocking part, and is capable of stabilizing a location thereof in the lumen by changing the dimension thereof.

11. The ultrasound medical apparatus of claim 1, further comprising a guiding hollow tube that is used for guiding the main body part, the first blocking part and the second blocking part into the lumen through the insertion opening, and is provided with a marker indicating the length of a part being inserted into the subject.

12. The ultrasound medical apparatus of claim 1, further comprising a mechanism configured for moving a movable part that includes at least the ultrasound transducer of the main body.

13. The ultrasound medical apparatus of claim 1, further comprising:
a determining part that determines amount of change of the dimension of at least one of the first blocking part and the second blocking part based on lumen size information prepared in advance; and
a controller that changes the dimension of at least one of the first blocking part and the second blocking part based on the amount of change determined by the determining part.

14. The ultrasound medical apparatus of claim 1, further comprising:
a first judging part that judges whether liquid is leaked out from the space between the first blocking part and the second blocking part based on a liquid supply state by the liquid supplying part; and
a first notifying part that performs notification based on judgment result obtained by the first judging part.

15. The ultrasound medical apparatus of claim 1, further comprising one or more cable parts whose one end is connected to each of the main body part, the first blocking part and the second blocking part, wherein
the one or more cable parts comprises:
a signal line configured for transmitting signals between the main body part and an external apparatus;
a member configured for changing the dimension of each of the first blocking part and the second blocking part; and
a member configured for supplying the liquid from the liquid supplying part into the space between the first blocking part and the second blocking part.

16. An ultrasound diagnosis apparatus comprising:
the ultrasound medical apparatus of claim 1; and
an image generating part that generates image data by processing signals based on echoes received by the ultrasound transducer.

17. The ultrasound diagnosis apparatus of claim 16, further comprising:
a second judging part that judges whether or not distribution of the liquid in the space between the first blocking part and the second blocking part is appropriate based on the image data generated by the image generating part; and
a second notifying part that performs notification based on judgment result obtained by the second judging part.

18. The ultrasound medical apparatus of claim 6, wherein
the liquid supplying part includes a tubular member that passes through the second blocking part, wherein an opening at a tip of the tubular member is exposed to the space between the first blocking part and the second blocking part, and
the liquid supplying part supplies the liquid to the space through the tubular member.

19. The ultrasound medical apparatus of claim 6, wherein the liquid supplying part supplies new liquid while collecting liquid that has already supplied to the space.

20. The ultrasound medical apparatus of claim 6, further comprising a stabilizer that is inserted into the lumen, is arranged at the opposite side to the insertion opening across the first blocking part, and is capable of stabilizing a location thereof in the lumen by changing the dimension thereof.

21. The ultrasound medical apparatus of claim 6, further comprising a guiding hollow tube that is used for guiding the main body part, the first blocking part and the second blocking part into the lumen through the insertion opening, and is provided with a marker indicating the length of a part being inserted into the subject.

22. The ultrasound medical apparatus of claim 6, further comprising a mechanism configured for moving a movable part that includes at least the ultrasound transducer of the main body.

23. The ultrasound medical apparatus of claim 6, further comprising:
a determining part that determines amount of change of the dimension of at least one of the first blocking part and the second blocking part based on lumen size information prepared in advance; and
a controller that changes the dimension of at least one of the first blocking part and the second blocking part based on the amount of change determined by the determining part.

24. The ultrasound medical apparatus of claim 6, further comprising:
a first judging part that judges whether liquid is leaked out from the space between the first blocking part and the second blocking part based on a liquid supply state by the liquid supplying part; and
a first notifying part that performs notification based on judgment result obtained by the first judging part.

25. The ultrasound medical apparatus of claim 6, further comprising one or more cable parts whose one end is connected to each of the main body part, the first blocking part and the second blocking part, wherein
the one or more cable parts comprises:
a signal line configured for transmitting signals between the main body part and an external apparatus;
a member configured for changing the dimension of each of the first blocking part and the second blocking part; and
a member configured for supplying the liquid from the liquid supplying part into the space between the first blocking part and the second blocking part.

26. An ultrasound diagnosis apparatus comprising:
the ultrasound medical apparatus of claim 6; and
an image generating part that generates image data by processing signals based on echoes received by the ultrasound transducer.

27. The ultrasound diagnosis apparatus of claim 26, further comprising:
a second judging part that judges whether or not distribution of the liquid in the space between the first blocking part and the second blocking part is appropriate based on the image data generated by the image generating part; and
a second notifying part that performs notification based on judgment result obtained by the second judging part.
